(19) United States
(12) Patent Application Publication
(10) Pub. No.: US 2013/0315834 A1
(43) Pub. Date: Nov. 28, 2013

(54) NANOPROBE COMPRISING GOLD COLLOID NANOPARTICLES FOR MULTIMODALITY OPTICAL IMAGING OF CANCER AND TARGETED DRUG DELIVERY FOR CANCER

(71) Applicants: Nagamani Praveen, Singapore (SG); Praveen Thoniyot, Singapore (SG); Malini Olivo, Singapore (SG)

(72) Inventors: Nagamani Praveen, Singapore (SG); Praveen Thoniyot, Singapore (SG); Malini Olivo, Singapore (SG)

(21) Appl. No.: 13/825,810

(22) PCT Filed: Sep. 23, 2011
(86) PCT No.: PCT/SG2011/000330

§ 371 Date: Aug. 12, 2013

(30) Foreign Application Priority Data
Sep. 24, 2010 (US) 61385977

(51) Int. Cl. A61K 49/00

(52) U.S. Cl. CPC A61K 49/0058
USPC 424/9.6; 435/7.23

(57) ABSTRACT

The present invention is directed to a nanoparticle loaded with a light sensitive molecule and a method of preparing the nanoparticle, wherein the nanoparticle is a colloidal gold nanoparticle and the light sensitive molecule is non-covalently adsorbed to the surface of the nanoparticle. The present invention is also directed to a nanoprobe comprising the nanoparticle and further comprising a targeting moiety covalently coupled to the surface of the nanoparticle. Additionally, the present invention is directed to an imaging method comprising administering the nanoprobe to a subject and collecting imaging data of the subject or part of the subject with optical multimodality imaging. A method of treating cancer in a subject comprising administering the nanoprobe and performing photodynamic therapy on the subject is further disclosed.

### CROSS-REFERENCE TO RELATED APPLICATION

This application makes reference to and claims the benefit of priority of an application for “Multimodality Optical Imaging of Cancer using Targeted, Drug Loaded Colloidal Gold Particles and their Application in Photodynamic and/or Photo thermal Therapy of Cancer” filed on Sep. 24, 2010 with the United States Patent and Trademark Office, and there duly assigned Ser. No. 61/385,977. The content of said application filed on Sep. 24, 2010 is incorporated herein by reference for all purposes, including an incorporation of any element or part of the description, claims or drawings not contained herein and referred to in Rule 20.5(a) of the PCT, pursuant to Rule 4.18 of the PCT.

### TECHNICAL FIELD

Various embodiments relate to the field of nanoprobes, in particular, nanoprobes for multimodality optical imaging and drug delivery.

### BACKGROUND

Cancer is one of the most prevalent diseases and accounts for around 13% deaths worldwide. Many cancers are usually diagnosed at a late stage which is bad for the patient's prognosis. Hence there is a need for developing an improved technology for early detection and treatment of cancer. Amongst the several treatments available, photodynamic therapy (PDT) is an alternative cancer treatment that has been gaining interest in recent years.

PDT is a minimally invasive technique that combines a light sensitive molecule called a photosensitizer (PS) that preferentially accumulates in the neoplastic tissue with light of specific wavelength to generate cytotoxic reactive oxygen species (ROS) that are toxic to cancerous cells resulting in tumor cell death by apoptosis or necrosis. The main advantages of PDT over conventional cancer treatments such as surgery, chemotherapy and radiotherapy are its minimal side effects, that no drug resistance can develop and its reduced toxicity that allows for repeated treatment. The effectiveness of PDT depends not only on the intracellular photosensitizer concentration, the intensity and the duration of light irradiation, but also on the subcellular localisation, the degree of aggregation, the photobleaching characteristics, and the singlet oxygen quantum yield of the photosensitizer. Therefore, it is important to develop a photosensitizer or improve an existing photosensitizer for targeted delivery to enhance the effectiveness of PDT.

One example of a photosensitizer is hypericin which has the ability to produce singlet oxygen upon activation with light. This makes it a very attractive candidate for PDT.

Hypericin, belonging to a class of class of naphthalene perylene dione anthrone compounds, or more specifically a phenanthroperylenequinone photosensitizer obtained from Hypericum perforatum (St. John's Wort) is a promising second-generation photosensitizer with good photosensitizing characteristics and tumor selectivity. Topical or systemic administration of hypericin results in a selective accumulation of hypericin in neoplastic tissue and thus it has great potential in fluorescence-guided diagnosis or photodynamic diagnosis of early neoplasms.

Hypericin is a hydrophobic molecule and the resulting insolubility in most of the physiologically acceptable media makes systemic administration of this photosensitizer problematic. Hypericin is usually dissolved in dimethylsulfoxide (DMSO) for in-vitro and animal use or ethanol for application in humans. Thus, to improve the solubility and increase the biological specificity of such hydrophobic photosensitizers for tumor cells, delivery systems based on liposomes, cyclodextrins, polymeric micelles, dendrimers and nanoparticles have been developed. This has led to the development of third-generation photosensitizers with the most promising results to date.

A newly emerging mode of delivery is a nanoparticle-based approach, where the photosensitizer is encapsulated in the nanoparticle or immobilized on the nanoparticle surface. The advantage of this approach is that the photosensitizer is delivered to the tumor site in a more selective manner with low toxicity and minimal damage to the normal tissues. This technique has further advantages due to the size tunability, surface characteristics and high drug loading capability of the nanoparticles. Biocompatible gold nanoparticles (GNPs) have great potential in clinical applications due to their easy preparation, efficient bioconjugation, non-cytotoxicity upon PEGylation and strong absorption and scattering properties, making them ideal candidates for contrast enhanced optical imaging. For example, hypericin has interesting Raman scattering properties. Because of the intrinsic Raman activity of hypericin, it may be used for Surface Enhanced Raman Spectroscopy (SERS) based imaging techniques upon immobilization on GNPs.

In addition to that, GNPs also contribute to photothermal effect that may be utilized in photothermal therapy (PTT).

So far, it has been reported that for a photosensitizer to be delivered into a cell, it needs to be attached to the GNP surface by a covalent anchoring method. This has the drawback that this covalent attachment may significantly alter the structure and activity of the photosensitizer.

Earlier reports on drug delivery using gold nanoparticles were based on covalent conjugation of the synthetic photosensitizer by functionalizing the photosensitizer with a thiol group or by covalent interactions of an amine group of the photosensitizer with the surface of colloidal gold. Such synthetic modifications may be detrimental to the activity of a drug molecule. Although these protocols show some success in delivery, the effect on the activity of the photosensitizer is still under debate.

Thus it is an object of the present invention to address at least the problems mentioned above and to provide a nanoprobe for multimodality optical imaging of cancer cells and targeted drug delivery to cancer cells.

### SUMMARY OF THE INVENTION

In a first aspect, the present invention relates to a nanoparticle loaded with a light sensitive molecule, wherein the nanoparticle is a colloidal gold nanoparticle and the light sensitive molecule is non-covalently adsorbed to the surface of the nanoparticle.

According to a second aspect, the present invention relates to a nanoprobe comprising the nanoparticle of the invention and further comprising a targeting moiety covalently coupled to the surface of the nanoparticle.

According to a third aspect, a pharmaceutical formulation comprising the nanoparticle or the nanoprobe of the invention is provided.

In a fourth aspect, a method of preparing a nanoparticle according to the invention comprising the steps of providing a colloidal gold nanoparticle, and non-covalently adsorbing a light sensitive molecule to the surface of the gold nanoparticle such that the light sensitive molecule is immobilized on said surface is provided.

In a fifth aspect, an imaging method comprising administering the nanoprobe according to the invention, and collecting imaging data of the subject or part of the subject with optical multimodality imaging is provided.

According to a sixth aspect, a method for determining a photodynamic therapy regimen for a subject comprising determining the therapy regimen based on imaging data collected with optical multimodality imaging after the nanoprobe or the nanoparticle according to the invention has been administered to the subject is provided.

According to a seventh aspect, the invention encompasses the use of intrinsic Raman activity of a light sensitive molecule for Surface Enhanced Raman Spectroscopy (SERS) based imaging, wherein the light sensitive molecule is a photosensitizer comprised in the nanoparticle or the nanoprobe of the invention.

In an eighth aspect, the invention provides a method of treating cancer in a subject comprising administering the nanoprobe according to the invention; and performing photodynamic therapy on the subject.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 shows a schematic diagram of a scheme for constructing or preparing a multimodality hypericin-gold theraganostic probe, in accordance to various embodiments;

FIG. 2 shows (a) a SEM image of the prepared GNP; and (b) an absorption spectrum of GNP, in accordance to various embodiments;

FIG. 3 shows a SERS spectra of Hypericin-GNP conjugate, in accordance to various embodiments;

FIGS. 4a(i) and 4a(ii) show dark field microscope images of control A-431 cells with no particles, in accordance to various embodiments;

FIGS. 4b(i) to 4b(iii) show dark field microscope images of A-431 cells with bioconjugated nanoparticles with hypericin, in accordance to various embodiments;

FIGS. 4c(i) to 4c(iii) show dark field microscope images of A-431 cells with pegylated nanoparticles with hypericin, in accordance to various embodiments;

FIGS. 4d(i) and 4d(ii) show dark field microscope images of A-431 cells with pegylated gold nanoparticles with hypericin, in accordance to various embodiments;

FIG. 5a shows OCT m-scans of (i) colloidal suspension of nanogold; (ii) 1% Intralipid used to mimic tissue scattering; (iii) colloidal suspension of naked 302 nm silica nanoparticles (2×10¹¹ particles/ml); and (iv) saline as a negative control, in accordance to various embodiments;

FIG. 5b shows OCT contrast agent on cancer cells with nanogold hypericin with (i) EGFR (Epidermal Growth Factor Receptor) fluorescence image; (ii) reflectance image of nanogold-hypericin; and (iii) combined images, in accordance to various embodiments;

FIG. 5c shows OCT reflectance based confocal images of A-431 EGFR positive cancer cells incubated with anti-EGFR labelled hypericin-GNP, in accordance to various embodiments;

FIG. 6 shows an exemplary OCT reflectance based confocal images of CNE2 cancer cells compared with normal NHFB cells incubated with anti-EGFR labelled hypericin-GNP, in accordance to various embodiments;

FIGS. 7a and 7b show TEM analysis of cell section of A-431 cells, in accordance to various embodiments.

FIG. 8 shows confocal fluorescence microscopy images (a) CNE2 cells treated with 2 μM hypericin for about 6 hours; and (b) A-431 cells treated with Hypericin-Au-EGFR theragnostic probe for about 6 hours;

FIG. 9a shows confocal fluorescence image showing uptake of hypericin after incubation with anti-EGFR conjugated hypericin GNP in A-431 cells

FIG. 9b shows a plot of comparison of hypericin fluorescence intensity in A-431 cells following incubation with anti-EGFR conjugated hypericin GNP and hypericin alone;

FIG. 10 shows a plot illustrating the dark toxicity of anti-EGFR conjugated hypericin GNP compared to hypericin at different concentrations ranging from 2-20 μM, in accordance to various embodiments;

FIG. 11 shows confocal fluorescence microscopy images after exposure to light for PDT in the case of A-431 cells treated with (a) Hypericin-Au-EGFR theragnostic probe, (b) Hypericin 2 μM; (c) Hypericin 4 μM; (d) Hypericin 6 μM; (e) Hypericin 8 μM; (f) Hypericin 10 μM; (g) A-431 cells treated with Hypericin-Au-EGFR theragnostic probe before PDT; and (h) A-431 cells control treated with no particles, in accordance to various embodiments;

FIG. 12 shows confocal image of A-431 cells undergoing apoptosis (as indicated by the white encircled area) and necrosis (as shown by the lighter shade indicated by the white arrow) following (a) PDT and (b) PTT, in accordance to various embodiments; and

FIG. 13 shows plots illustrating percentage of cell death following (a) PDT and (b) PTT using different concentration of anti-EGFR conjugated hypericin GNP and anti-EGFR GNP, respectively, in accordance to various embodiments.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not, necessarily mutually exclusive, as some embodiments can be combined with one or more other embodiments to form new embodiments.

In a first aspect, a nanoparticle loaded with a light sensitive molecule, wherein the nanoparticle is a colloidal gold nanoparticle and the light sensitive molecule is non-covalently adsorbed to the surface of the nanoparticle is provided.

In the context of various embodiments, the term “nanoparticle” may refer to an object of a size less than about 1 micron or 1 μm.

For example, the gold nanoparticle may be about 10 nm to about 1000 nm in size. In various embodiments, the gold nanoparticle may be about 40 nm in size. Depending on the shape of the nanoparticle, the size relates to the diameter or length of the respective structure. In various embodiments, the size is the mean particle size. A gold nanoparticle may be selected from the group consisting of a gold nanosphere, a gold nanorod, a gold nanotube, a gold nanoshell, a gold nanodot and a gold nanowire. Although gold nanorods have generally been identified to have superior photothermal effects because of their near infrared (NIR) absorption, uptake of spherical gold particles is usually better than that of gold nanorods. Additionally, the toxicity of surfactants, for example, cetyltrimethylammonium bromides (CTAB), on the surface of gold nanorods may limit their applications. As the uptake of a gold nanoparticle depends significantly on its size and shape, maximum uptake into the cells may be achieved by using spherical gold nanoparticles with a mean diameter of about 40 nm. In some embodiments the nanoparticles are essentially monodisperse.

As used herein, the term “colloidal gold nanoparticles” refers to gold nanoparticles capable of forming a colloid. A colloid may be analogous to a solution: both are systems of molecules, atoms or particles in a solvent. The nanoparticles of a colloidal system, however, because of their size (typically in nanometers) or the distance between them (also typically in nanometers), and their solid cores, may attract one another with sufficient force to make them tend to aggregate even when the only means of transport for the nanoparticles in the solvent is diffusion. A “colloidal gold nanoparticle” may not be itself a colloid but rather only a constituent of a colloid. Nonetheless, the term “colloid” may be used to denote the nanoparticle itself.

Colloidal gold nanoparticles may be coated and stabilized using different thiol ligands capped by chemical groups which provide a variety of solubilities to give the solubility properties of hydrophilic, hydrophobic, and amphiphilic. These gold nanomaterials are therefore suitable for a wide range of applications in different systems and environments, for example, acting as a delivery vehicle as well as a contrast agent.

Generally, gold is biocompatible with little or no long term toxicity and tunable in size/shape and aspect ratios. Gold has excellent optical properties, easy surface functionalization, possibility of bioconjugation, ability to adsorb hydrophobic drugs on the surface and tunable photothermal properties.

In the context of various embodiments, the term “non-covalently adsorbed” may generally relate to a non-covalent interaction which is a type of chemical interaction or bonding, typically between macromolecules, that does not involve the sharing of pairs of electrons, but rather involves more dispersed variations of electromagnetic interactions. There are four commonly mentioned types of non-covalent interactions, namely, hydrogen bonds, ionic bonds, van der Waals forces, and hydrophobic interactions. In various embodiments, the non-covalent interaction may be a hydrophobic interaction. Hydrophobic interaction may retain the activity of the light sensitive molecule unaltered, as there is no structural change involved in the adsorption process. Thus, this type of hydrophobic adsorption maintains the integrity of the photosensitizer in terms of structure and activity for its use in photodynamic therapy (PDT).

This is in contrast to a covalent interaction, which causes chemical modification to alter light sensitive molecule activity. Furthermore, with non-covalent interactions, the release of the light sensitive molecule from the gold nanoparticle (GNP) might be easier when compared to covalent interactions.

The term “light sensitive molecule” may generally be referred to as a molecule that is responsive to a light stimulus. This responsiveness includes chemical as well as physical changes of the molecule properties.

In various embodiments, the light sensitive molecule may be a photosensitizer. In the context of various embodiments, the term “photosensitizer” may generally refer to a molecular or atomic species that initiates a photochemical reaction. More specifically, it may relate to agents which, when stimulated by light, react with oxygen in the affected material to produce reactive oxygen species. Reactive oxygen species may be any chemical form of oxygen that is more chemically reactive than stable molecular oxygen (“triplet oxygen”). Such species include but are not limited to “singlet oxygen” (i.e., molecular oxygen in either of its two metastable states), and free oxygen radicals.

Exemplary photosensitizers include, but are not limited to hypericin, Photofrin, Visudyne, aminolevulinic acid-induced protoporphyrin IX (ALA-induced Pp IX), Foscan, Chorin e6, mono-L-aspartyl chlorin e6 (NPe6) or Laserphyrin, propiophenone, anthrone, benzaldehyde, butylophenone, 2-naphthylphenylketone, 2-naphthaldehyde, 2-acetonaphthone, 1-naphtylphenylketone, 1-acetonaphthone, 1-naphtho aldehyde, fluorenone, 1-phenyl-1,2-propane dione, benzoethrile, acetone, biacetyl, acridine orange, acridine, Rhodamine-B, eosine, fluorescein, Silicon Phthalocyanine Pc 4, m-tetrahydroxyphenylchlorin (mTHPC), Allumera, Levulan, Metvix, Amphinex, Azadipyrromethenes, and a mixture thereof.

In various embodiments, a photosensitizer may produce singlet oxygen upon activation with a light source. The light source may be a laser or a light emitting diode (LED). The light source may have a wavelength in the range from about 300 nm to about 800 nm.

In a second aspect, a nanoprobe comprising the nanoparticle according to the invention and further comprising a targeting moiety covalently coupled to the surface of the nanoparticle is provided.

In the context of various embodiments, the term “nanoprobe” may generally refer to an ultra-sensitive (optical) device that is used as a sub-cellular detection tool. More specifically, it comprises very small particles that may be used in the detection, diagnosis, and treatment of cancer.

In various embodiments, the targeting moiety may be selected from the group consisting of a small molecule, an antibody, an antigen, an affibody, a peptide, an aptamer, a cell surface receptor ligand, a nucleic acid, a fibronectin, a protein, a fusion protein, a peptide, a biotin, and conjugates thereof. Also included as targeting moieties are antibody-like molecules, antibody fragments and antibody derivatives, including but not limited to single chain antibodies, minibodies, diabodies, lipocalin muteins, Spiegelmers, and the like. The targeting moiety may also be a chemical moiety, for example an organic molecule, such as a small organic molecule. In other embodiments, the chemical moiety may be a metal complex.

The targeting moiety may be covalently coupled to the surface of the nanoparticle by a linking moiety. For example, the linking moiety may be polyethylene glycol (PEG) or a derivative thereof. In other embodiments, the linker moiety is a hydrocarbon, preferably a linear hydrocarbon chain with 2-20 carbon atoms. In still further embodiments, the linker is another polymer, such as a poly amino acid, PLA, PLGA or the like. In still further embodiments, the linker is a organic molecule with two functional groups, such as a diamine, dithiol, dicarboxylic acid and the like. In the context of various embodiments, the term “linking moiety” may be interchangably referred to as “linker moiety” or “linker”.

In various embodiments, the linker is PEG having a molecular weight ranging from about 1000 to about 8000, or ranging from about 3000 to about 5000. The term “PEG”, as used herein, includes PEG derivatives, such as carboxy PEG.

In various embodiments, the nanoprobe may be a multimodal optical nanoprobe. As used herein, the term “multimodal” may refer to having or involving several modes, modalities, or maxima. The term “modes” may generally refer to patterns, properties, functions and/or conditions in which two or more different methods, processes or forms of delivery are used. In this context, the terms “modalities” and “modes” may be used interchangably and relate to the property of being detectable by different techniques. More specifically, exemplary “modes” may be optical modes such as coherence tomography or fluorescence or brightfield or polarized light or darkfield or phase contrast, or other transmission wave modes such as ultrasonography. The nanoprobe may serve as multimodality optical imaging theragnostic probe for diagnosis in at least three optical modalities as well as photothermal therapy of cancer.

In a third aspect, a pharmaceutical formulation comprising the nanoparticle or the nanoprobe according to the invention is provided. Such a pharmaceutical composition can further comprise pharmaceutically accetable carriers and/or auxiliaries. Suitable agents are known to those skilled in the art and are for example described in Remington's Pharmaceutical Sciences (18^{th} ed.; Mack Publishing Co.; Easton), which is incorporated herein by reference in its entirety.

In accordance to a fourth aspect, a method of preparing a nanoparticle comprising the steps of providing a colloidal gold nanoparticle, and non-covalently adsorbing a light sensitive molecule to the surface of the gold nanoparticle such that the light sensitive molecule is immobilized on said surface is provided.

In various embodiments, the step of non-covalent adsorbing may comprise adding a solution of the light sensitive molecule into a solution comprising the colloidal gold nanoparticle, and sonicating the resulting mixture for about 2 hours at a temperature of about 20° C.

In various embodiments, the method may further comprise the step of functionalizing the nanoparticle with a targeting moiety. As used herein, the term “functionalizing” may be referred to as adding functional groups onto the surface of a material by chemical synthesis methods. A functional group added may be subjected to ordinary synthesis methods to attach virtually any kind of organic compound onto the surface. Functional groups are specific groups of atoms within molecules that are responsible for the characteristic chemical reactions of those molecules and may also be used to covalently link molecules such as fluorescent dyes, nanoparticles, proteins, DNA, and other compounds of interest for a variety of applications such as sensing.

In these embodiments, the targeting moiety may be as defined above.

Various embodiments provide the method, wherein the functionalizing step may comprise modifying the surface of the gold nanoparticle with a linker moiety and coupling the targeting moiety to the linker moiety. For example, the linker may be polyethylene glycol (PEG) or a derivative thereof. The PEG may be a PEG derivative, such as carboxy PEG. The linker may also be selected from the other exemplary linkers disclosed herein.

In various embodiments, the linker is carboxy PEG and the functionalizing step further comprises activating the carboxyl group of the carboxy PEG with N-(3-dimethylaminopropyl)-N′ ethylcarbodiimide (EDC) and N-Hydroxysuccinimide (NHS) to form O-acylisourea as an active ester, and reacting the active ester with amino groups on an antibody to covalently couple the antibody to the surface of the gold nanoparticle to form a bioconjugated gold nanoparticle.

As used herein, the term “carboxyl group” refers to a —COOH group. The term “amino group” generally refers to the functional group —NH₂. The terms “amine,” “hydroxy,” and “carboxyl” include such groups that have been esterified or amidified. Procedures and specific groups used to achieve esterification and amidification are known to those of skill in the art and can readily be found in reference sources such as Greene and Wuts, Protective Groups in Organic Synthesis, 3rd Ed., John Wiley & Sons, New York, N.Y., 1999, which is incorporated herein in its entirety.

The term “ester” refers to a class of organic compounds formed from an acid and an alcohol.

The term “bioconjugated gold nanoparticle” refers to a gold nanoparticle coupled to a biomolecule by a covalent linkage.

Generally, a colloidal dispersion can be a hydrophobic dispersion or a hydrophilic dispersion, wherein the hydrophobic dispersion may be thermodynamically unstable if the dispersion medium (or continuous phase) is aqueous, and wherein the hydrophilic dispersion may be unstable if the dispersion medium is a non-polar solvent.

In various embodiments, the method may further comprise adding a stabilizer prior to the reacting step. The stabilizer may comprise a sodium salt, for example, sodium azide.

Prior to the reacting step, the method may further comprise performing a separation step, for example a dialysis step, to remove unreacted reactants, such as EDC and NHS, and/or to remove the stabilizer.

In various embodiments, the targeting moiety is an antibody. The antibody includes but is not limited to an anti-EGFR (Epidermal Growth Factor Receptor) antibody or an anti-Her2Neu (Human Epidermal growth factor Receptor 2) antibody. In other embodiments, the antibody may be any suitable antibody that targets a biomarker for a disease of interest.

In the method of preparing the nanoparticle according to various embodiments, the nanoprobe may be properly functionalized using PEG and covalent conjugation of the anti-EGFR antibody, anti-Her2Neu, or anti-EGFR affibody by amide chemistry.

In a fifth aspect, an imaging method comprising administering the nanoprobe, and collecting imaging data of the subject or part of the subject with optical multimodality imaging is provided.

Generally, a multimodal imaging system is a medical imaging system that combines optical, radioactive and magnetic imaging modes. This method of imaging may include modes such as positron emission topography, optical fluorescence and bioluminescence as well as magnetic resonance spectroscopy and single photon emission topography. For example, multimodal imaging combines elements of MRI and PET scans as well as imaging tests with radioactive elements that illuminate imagery inside a body. Different methods may be used to study human tissue at the same time; thereby allowing medical doctors to see multiple aspects of the same area, for example, to see anything present in that specific tissue: its size, its exact location and its metabolic activity. This would then allow for analysis of the metabolic activity of surrounding tissues and evaluation of abnormalities or changes in the function of those tissues as a result of a condition or a tumor or any other medical complication.

In various embodiments, the optical multimodality imaging may be in vivo imaging or ex vivo imaging. Exemplary optical multimodality imaging may be selected from the group consisting of magnetic resonance imaging, ultrasound imaging, confocal fluorescence endomicroscopy, optical coherence tomography (OCT), Surface Enhanced Raman Spectroscopy (SERS) and a combination thereof.

In various embodiment, the portion of the subject from which the imaging data is collected comprises a tumor cell. For example, the tumor cell may be a cancer cell or a cancerous cell line.

For the imaging method according to various embodiments, by careful tuning of size and shape of colloidal gold, the nanoprobe may be used for deep tissue imaging using optical coherence tomography, NIR imaging with modified gold nanoparticles such as rods and shells and also fluorescence based imaging due to the photosensitizer fluorescence, for example, in hypericin. Thus, changing the size and shape of colloidal gold may enhance the utility of colloidal gold for imaging of deeply seated tumors.

In a sixth aspect, a method for determining a photodynamic therapy regimen for a subject, comprising determining the therapy regimen based on imaging data that has been collected with optical multimodality imaging after the nanoprobe or the nanoparticle has been administered to the subject is provided. The photodynamic therapy (PTD) program may be coupled with photothermal (PTT) effects provided by plasmonic heating effects of the nanoparticle. As used herein, the term “photothermal effect” may generally refer to a phenomenon associated with electromagnetic radiation involving the photoexcitation of material resulting in the production of thermal energy (heat). PTD may, for example, be used during treatment of blood vessel lesions, laser resurfacing, laser hair removal and laser surgery.

In a seventh aspect, a method for Surface Enhanced Raman Spectroscopy (SERS) based imaging using the intrinsic Raman activity of a light sensitive molecule, wherein the light sensitive molecule is a photosensitizer comprised in the nanoparticle or the nanoprobe, is provided.

In an eighth aspect, a method of treating cancer in a subject comprising administering the nanoprobe; and performing photodynamic therapy on the subject is provided. The photodynamic therapy may comprise incubating the nanoprobe with a tumor cell to allow internalization in the cell, and upon internalization, illuminating the cell to cause cell death by reactive oxygen species generated by the light sensitive molecule of the nanoprobe.

The term “reactive oxygen species” may be interchangably referred to as “oxygen radicals” or “pro-oxidants”, which are molecules or ions formed by the incomplete one-electron reduction of oxygen. These reactive oxygen intermediates may include singlet oxygen, superoxides, peroxides, hydroxyl radicals, and hypochlorous acid. Generally, reactive oxygen species contribute to the microbicidal activity of phagocytes, regulation of signal transduction and gene expression, and the oxidative damage to nucleic acids, proteins, and lipids.

In method of treating cancer according to the various embodiments, the light sensitive molecule may be a photosensitizer. The method may be based on targeting of a receptor in or on the surface of the cell. The receptor may, for example, be selected from the group consisting of an integrin, a somatostatin receptor, an epidermal growth factor receptor (EGFR), a Her-2/neu receptor, a glucose transporter (GLUT), a folate receptor, and a steroid receptor.

In the context of various embodiments, the term “about” or “approximately” as applied to a numeric value encompasses the exact value and a variance of +/−5% of the value.

The phrase “at least substantially” may include “exactly” and a variance of +/−5% thereof. As an example and not limitation, the phrase “A is at least substantially the same as B” may encompass embodiments where A is exactly the same as B, or where A may be within a variance of +/−5%, for example of a value, of B, or vice versa.

In order that the invention may be readily understood and put into practical effect, particular embodiments will now be described by way of examples and not limitations, and with reference to the figures.

### EXAMPLES

Bioconjugated nanosensitizers or nanoprobes such as optical and therapeutic probes for the detection, monitoring and treatment of cancer are explored. Various studies are carried out with hypericin immobilized onto the gold nanoparticle surface by hydrophobic and non-covalent interactions. Such hydrophobic and non-covalent interactions may retain the activity of hypericin unaltered, as there is no structural change involved in the adsorption process. This is an approach for the delivery of hypericin using gold nanoparticles as a carrier.

Moreover, surface of gold nanoparticle may be functionalized in the desired way. Here, the gold surface is pegylated with bifunctional PEG of which one end is covalently attached to gold and other end is available for functionalization that is used for attaching antibody for targeting. Targeting reduces the toxicity of the drug by increasing selectivity. Hence, targeted nanoparticles carrying load of photosensitizer when used for in vivo or in vitro (i.e., ex vivo) studies selectively accumulate in the tumor cells. Upon internalization of the nanoparticles with payload by illumination with appropriate wavelength, tumor cells undergo cell death by the reactive oxygen species generated by the photosensitizer.

In addition to the therapeutic effects, the nanoprobes may be developed as optical probes for multi-modality in vivo optical imaging technology such as in vivo 3D confocal fluorescence endomicroscopic imaging, optical coherence tomography (OCT) with improved optical contrast using nano-gold and Surface Enhanced Raman Scattering (SERS) based imaging and bio-sensing. These techniques may be used in tandem or independently as in vivo optical biopsy techniques to specifically detect and monitor specific cancer cells in vivo. Such nanoprobe-based optical biopsy imaging technique has the potential to provide an alternative to tissue biopsy and will enable clinicians, for example, medical doctors to make real-time diagnosis, determine surgical margins during operative procedures and perform targeted treatment of cancers.

Generally, gold nanoparticles based drug delivery, for example, in PDT has advantages such as reduced toxicity, ability to surface functionalize and bioconjugate, tenability of size and shape, photothermal effects and imaging capability in multimodal platforms.

In the following examples, the use of antibody conjugated hypericin loaded GNPs (nanoprobes) for multi-modality optical diagnostic imaging and therapy of cancer is studied. The conjugation of photosensitizer to GNP results in improved effectiveness of destroying tumor cells. This combination of nanotechnology and photodynamic therapy may provide impetus to widen its scope as an alternative method for multimodality imaging and treatment of cancer.

### Scheme for Constructing Multimodality Hypericin-Gold Theraganostic Probe

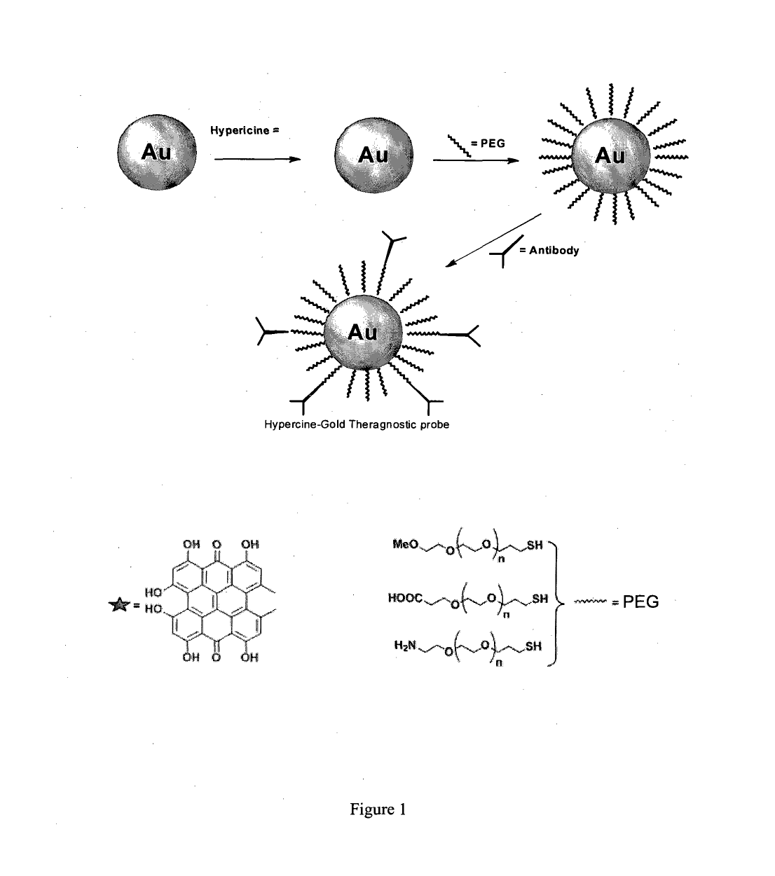

FIG. 1 shows a schematic diagram of the scheme for constructing or preparing a multimodality hypericin-gold theraganostic probe by non-covalent absorption of hypericin on colloidal gold. This scheme may be general for any hydrophoic PDT drug.

Various components to the hypericin-gold theragnostic probe attribute to different functions. Appropriate antibody conjugation allows for selectivity to cancer cells. By selection of gold concentration, particle size and shape, drug loading may be controlled. In the scheme, the ability to load the drug on to gold without chemical modification allows for unaltered therapeutic activity. The intrinsic fluorescence of the drug provides for optical imaging by confocal endomicroscopy, while the gold nanoparticle provides for optical coherence tomography. The intrinsically high Raman cross-section of the photosensitizer allows for Surface Enhanced Raman Spectroscopy (SERS).

Table 1 shows the role of different entities in the PDT drug delivery system as seen in the scheme of FIG. 1.

TABLE 1

| Entity | Representation | Roles |
|---|---|---|
| Gold nanoparticle | 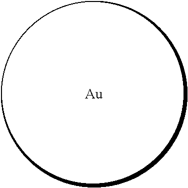 | Imaging Hyperthermic Effect SERS Activity Multiplexing capability No toxicity |
| Hypericin | 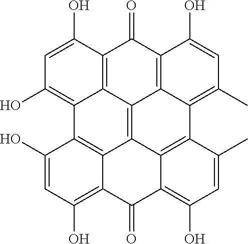 [SMILES: Cc1cc(O)c2c(=O)c3c(O)cc(O)c4c5c(O)cc(O)c6c(=O)c7c(O)cc(C)c8c1c2c(c34)c(c78)c65] | Photo Dynanimc drug SERS reporter |
| PEG | 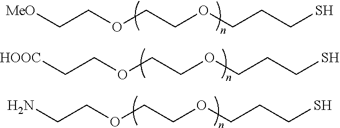 | Protection of Au Stabilization of SERS End group for bioconjugation No Biofouling Reduced toxicity |
| Antibody (e.g., Anti-HER-2 neu or Anti-EGFR) | 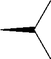 | Active Targeting |

Photodynamic therapy of cancer is governed by the drug molecule, while photothermal treatment after optical biopsy is attributed to the gold nanoparticles.

### Methodology

General steps involved in the prepartion of a hypericin-gold theragnostic probe are as follow:

Step 1. Synthesis of gold nanoparticles of appropriate size and shape using UV spectroscopy and transmission electron microscopy (TEM) for Surface Plasmon Resonance (SPR) and size characterization;
Step 2. Immobilization of hypericin on gold nanoparticles using UV spectroscopy to ensure that gold is not aggregated, and SERS to ensure hypericin is attached to nanoparticles.
Step 3. PEGylation of gold nanoparticle+hypericin complex using polyethylene glycol (PEG) with desired functionalities.
Step 4. Activation of the carboxy end groups on the carboxy PEG with EDC [N-(3-dimethylaminopropyl)-N′ ethylcarbodiimide] and NHS [N-Hydroxysuccinimide] and dialysis of activated gold nanoparticles+hypericin+PEG complex to remove unreacted EDC and NHS.
Step 5. Bioconjugation of the antibody to the active ester intermediate for successful attachment of antibody for targeting.
Step 6. Purification of the biconjugated gold nanoparticles loaded with photosensitizer, to remove unreacted antibody by centrifugation using UV spectroscopy for ensuring no aggregation, and SERS to ensure hypericin is still attached to gold even after pegylation and rigorous centrifugation steps involved in purification.
Step 7. Incubation of bioconjugated nanoparticles with tumor cells to allow the nanoparticles to internalize in the cells.

Preparation of GNPs:

All the glassware are washed with aqua regia (3HCl: 1 HNO₃) and thoroughly rinsed with milli q water and oven dried to remove artifical nucleation sites. Briefly, a 200 ml solution of 0.259 mM tetrachloroauric acid (HAuCl₄) is brought to about 90° C. before a 3 ml solution of 34 mM trisodium citrate is added rapidly to the HAuCl₄ solution, while maintaining vigorous stirring throughout the process. The mixture is heated continuously at about 90° C. for about the next 10 mins under stirring before a color change from gray to ruby red (or burgundy) is observed, indicating the formation of the GNPs. Intermediate colour changes of gray to black to purple before arriving at ruby red may also be observed. Thereafter, heating is stopped while continuing stirring and solution is made up to final volume of 200 ml with milli q water, stored in a bottle and protected from light. Particles are stable under refrigeratration conditions for a period of about 8 months. UV absorbance is recorded and particle size is characterized by TEM analysis. By this preparation procedure, particles of about 40 nm in size are obtained.

Immobilization of Hypericin:

500 μl of 100 mM hypericin in DMSO is added drop wise to 5 ml GNP solution and sonicated for about 2 hours at about 20° C. Sonication may be carried out with occasional cooling of a bath. A SERS spectrum analysis may be used to confirmed the immobilization of hypericin on the GNP.

Pegylation:

Hypericin immobilized GNPs (about 5 ml) are pegylated using 1 μM of heterofunctional PEG (PEG-SH—COOH, 3000 kDa) by vigorous mixing for 15 mins followed by the addition of about 3 ml, 10 μM HS-PEG-OMe (5000 kDa) under constant stirring for about 30 mins for complete coverage. After complete pegylation, excess PEG is removed by centrifugation and resuspension in phosphate-buffered saline (PBS pH 7.4). The centrifugation may be carried out in 3 rounds. UV measurements are carried out indicating no aggregation and SERS spectrum shows the presence of hypericin even after the rigorous centrifugation steps.

Bioconjugation of the Anti-EGFR:

About 5 ml of resuspended pegylated GNPs is treated with 40 mg/ml N-(3-dimethylaminopropyl)-N′ ethylcarbodiimide (EDC) and 110 mg/ml N-hydroxysuccinimide (NHS) by vigorous mixing for about 15 mins at room temperature to form an active ester. Unreacted EDC and NHS are removed by filtering using Nanosep filters. 200 mg/ml anti-EGFR antibody (Santacruz, USA) is then added and incubated for about 2 hours at about 4° C. Unconjugated antibodies are removed by filtering with Nanosep filters. Antibodies may be dialyzed to remove sodium azide that has been used as a stabilizer prior to the addition to the active ester. Dialysis is carried out using spectrapor dialysis membranes with appropriate molecular weight cut off.

Characterization of Nanoprobes:

The particle size of the nanoprobes is determined by scanning electron microscopy (JEOL, JSM-5200, Japan) and their UV-V is extinction spectrum was characterized using a UV-V is spectrophotometer (Shimadzu UV-2401 PC).

SERS Measurements:

The SERS characterization of the nanoprobe is carried out by Renishaw InVia Raman (UK) microscope system having an excitation laser at 633 nm About 20 μl of the nanoprobe solution containing at least 2.6×10¹° particles/ml is placed on a glass slide and covered with a cover slip to perform Raman measurements.

Cell Culture:

A-431, a human epidermoid carcinoma cell line over expressing wild-type EGFR, is obtained from American Type Culture Collection (ATCC). The cells are cultured as a monolayer in Dulbecco's Modified Eagle Medium (DMEM) medium supplemented with 10% fetal bovine serum,1% non-essential amino acids (Gibco, USA), 1% sodium pyruvate (Gibco, USA), 100 units ml-1 penicillin/streptomycin (Gibco, USA) and incubated at about 37° C., 95% humidity and 5% CO₂. Tumor cells may be trypsinized to remove the adhesive cell culture and cell count may be performed with trypan blue and hemocytometer.

Cell Localization Studies:

Targeting and localization of the nanoprobes to cellular receptors is assessed using dark field contrast imaging and confocal fluorescence microscopy. Briefly, A-431 cells are incubated with the nanoprobes for about 3 hours at about 37° C. The cells are then washed in PBS, fixed in 2% paraformaldehyde for about 10 mins and mounted in Vectashield fluorescent mounting medium (Vector Laboratories, Burlingame, Calif.). The cells are visualized via dark field illumination system (CytoViva, Auburn, Ala.) attached to a Nikon Eclipse 80i microscope. The in-vitro localization of the nanoprobes is also assessed by exciting the cells using a 488 nm argon laser. Fluorescence emission in the wavelength range of 590-630 nm is split by a dichroic filter and detected through a band pass filter (BP-610 nm; Omega Optical, Brattleboro, Vt.). Post-capture image analysis is performed using ImageJ 1.42q (National Institutes of Health) software.

Toxicity Assay:

To assess the cell viability, A-431 cells are counted and seeded at 5×10³ cells per well into 96-well plates and allowed to adhere for about 24 hours at about 37° C. Cells are treated with various concentrations (2-20 μM) of nanoprobes for about 24 hours in triplicates. Thereafter, medium is decanted and a colorimetric cell viability MTT assay is performed according to the manufacturer's protocol (in-vitro toxicology assay kit, TOX-1, Sigma). Briefly, about 150 μl of MTT reconstituted in Hanks Balanced Salt Solution (HBSS) is added into each well and incubated for about 2 hours at about 37° C. About 150 μl of solubilisation solution is added to dissolve the MTT formazan crystals. Absorbance is measured at 570 nm using 630 nm as the reference filter. Absorbance given by untreated cells is taken as 100% cell growth.

In-Vitro Photodynamic Therapy (PDT):

A-431 cells are seeded at 2×10⁴ cells per well into 8-well chambered slides and allowed to adhere overnight at about 37° C. The culture medium is then removed and replaced with fresh serum free medium with different concentrations of nanoprobe (5-10 μM). After about 3 hours of incubation, the medium is again replaced by fresh medium and the cells are irradiated at a fluence of 1 J/cm². Following incubation in the dark for about 18 hours, the irradiated cells are evaluated for apoptosis and necrosis using the PromoKine Apoptotic/Necrotic/Healthy Cells Detection Kit (Heidelberg, Germany).

In Vitro Photothermal Cell Studies:

To assess the photothermal effect (PTT) of the GNPs used in the nanoprobes, the cells are incubated with medium containing different concentrations (5-10 μM) of EGFR antibody conjugated GNPs (without hypericin) for 45 mins. Following, incubations cells are irradiated at a fluence of 1 J/cm². After about 18 hours of incubation, the PTT treated cells are assessed for apoptotic and necrotic cell death using the Promokine apoptotic and necrotic cell detection kit (Heidelberg, Germany).

### Example 1

### SEM of nanoprobes

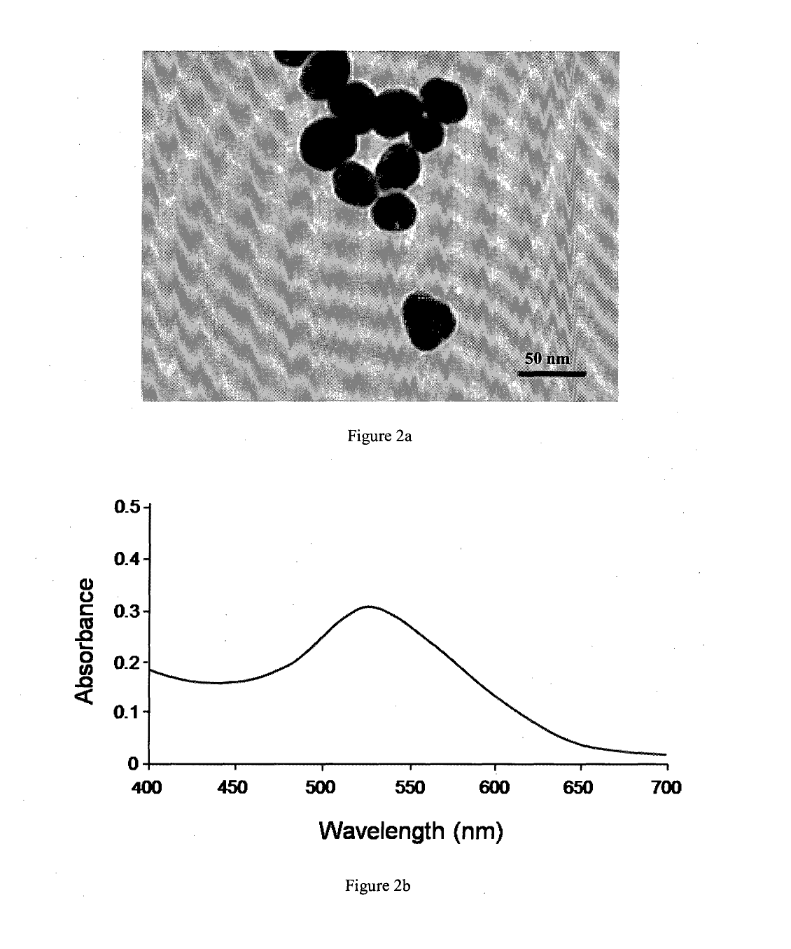

The SEM image of the synthesized GNPs is shown in FIG. 2a. The image shows that the particles are spherical and with an average diameter of 40 nm. The absorption maxima of GNPs are located at 528 nm (FIG. 2b) which corresponds to the surface plasmon resonance typical of GNPs of that size.

### Example 2

### SERS Measurement of Hypericin Nanoprobe

The key property for any nanoprobe for targeted PDT would be the ability to load enough quantity of photosensitizer on to the gold nanoparticle surface without losing its activity. The best method for loading a compound without losing the activity would be via non covalent interactions which will eliminate the need of chemical modification of the drug molecule. Surface spectroscopy techniques may be used to study such a loading of photo sensitizer.

The SERS technique allows to monitor the hypericin behavior in the complex via a strong enhancement of the intensity of Raman signal from hypericin vibrations and the quenching of its fluorescence caused by proximity of this molecule to the GNP. SERS may be used as characterization technique to provide proof of hypercine incorporation in the Hypericin-Au-EGFR construct.

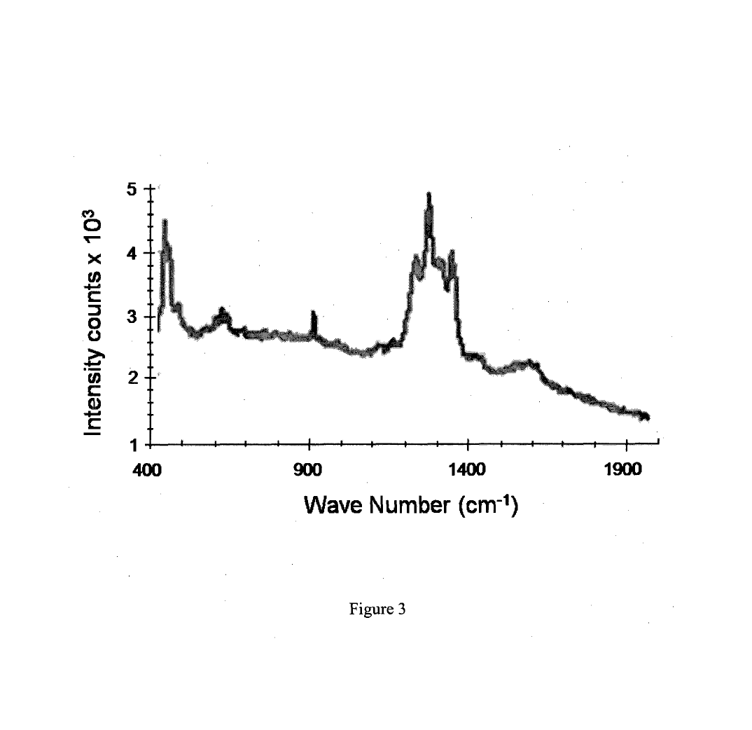

FIG. 3 shows a SERS spectrum of Hypericin-Au-EGFR theragnostic probe recorded at 633 nm laser with power of 3.15 mW under physiological conditions using 1 Os acquisition time. The SERS spectrum of the construct shows envelop of signals typical of hypercine between 1000 and 1500 cm⁻¹. These peaks are the Raman scattering of hypericin that is enhanced by the gold nanoparticle to which the hypericin is attached to. From this spectrum it nay be concluded that, hypericin is immobilized on the gold nanoparticles. The strong SERS spectrum of hypericin in Hypericin-Au-EGFR indicate the possibility of SERS based biosensing, for example, SERS based imaging using the probe for cancer detection.

### Example 3

### In-vitro localization of nanoprobe

In-vitro localization of anti-EGFR labelled hypericin-GNP shows internalization of the particles within about 3 hours in A-431 epidermoid cancer cells via dark field microscopy.

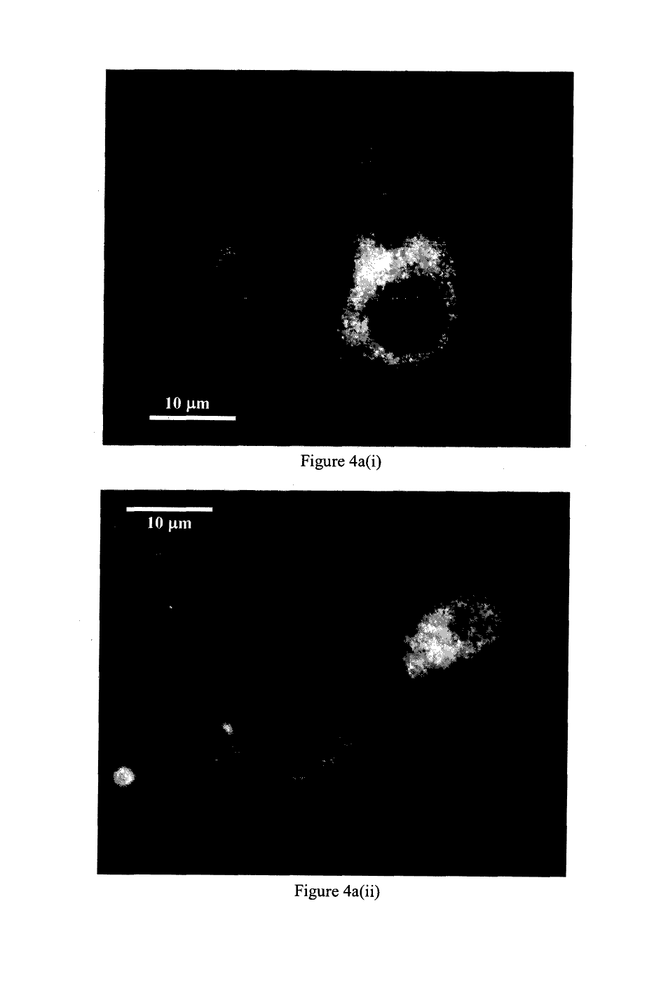

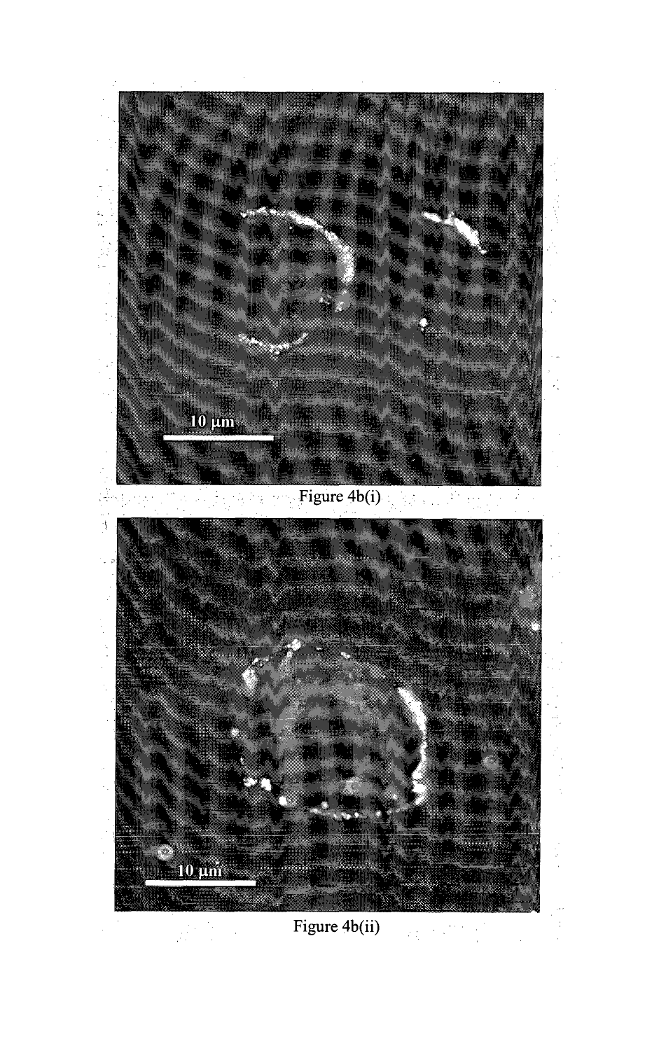

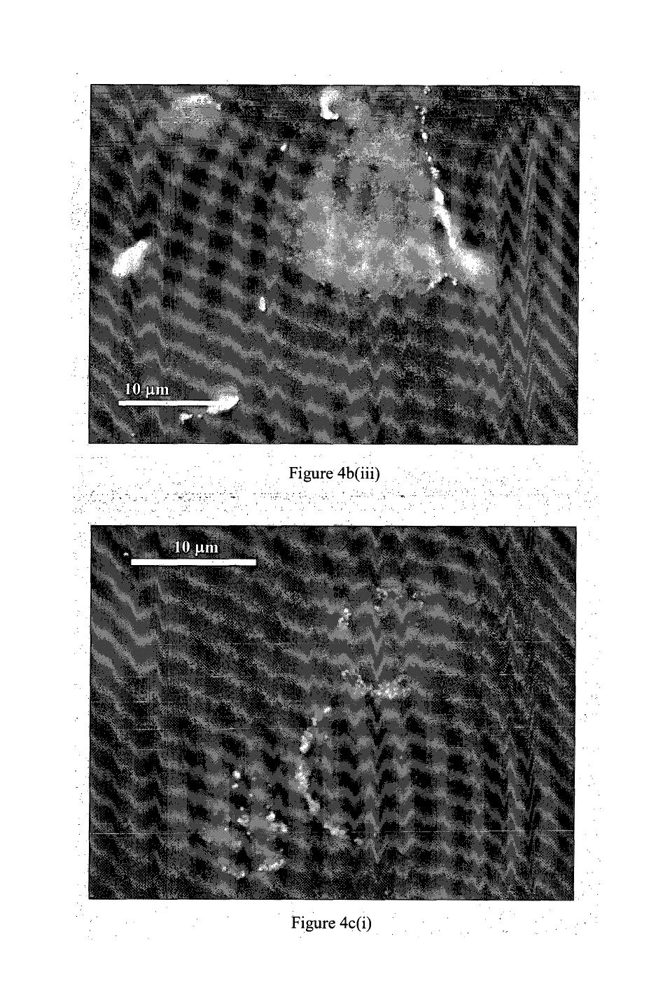

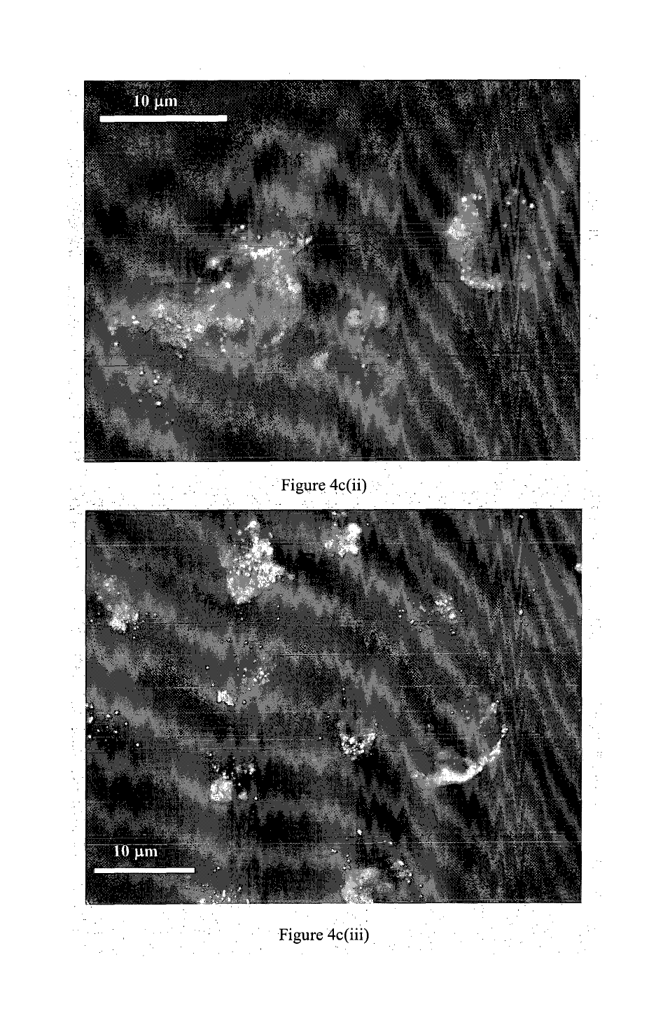

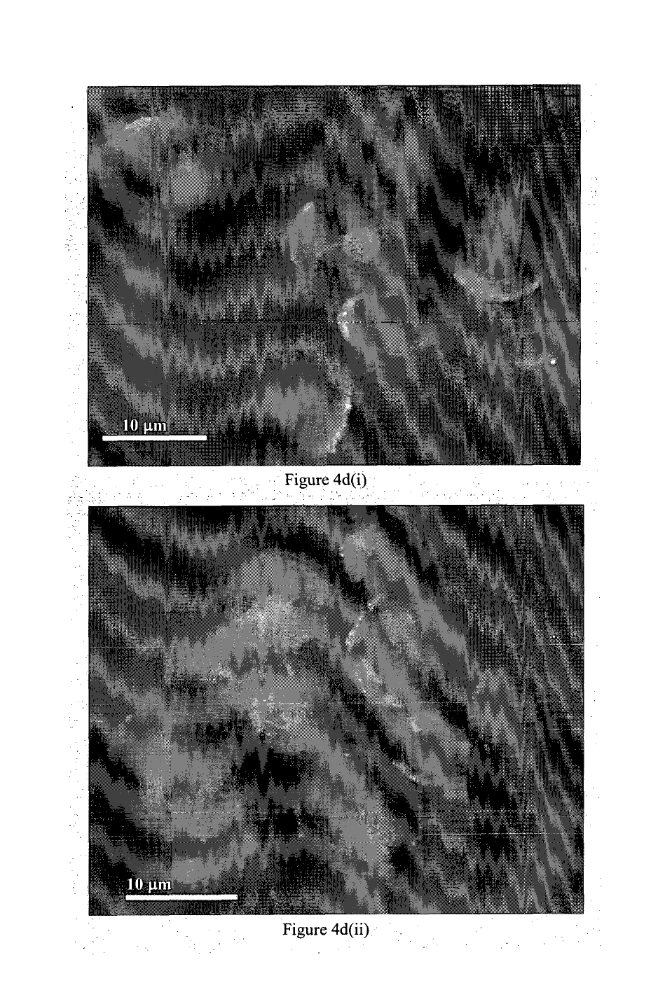

FIGS. 4a(i) and 4a(ii) show dark field microscope images of control A-431 cells with no particles. FIGS. 4b(i) to 4b(iii) show dark field microscope images of A-431 cells with bioconjugated nanoparticles with hypericin. FIGS. 4c(i) to 4c(iii) show dark field microscope images of A-431 cells with pegylated nanoparticles with hypericin. FIGS. 4d(i) and 4d(ii) show dark field microscope images of A-431 cells with pegylated gold nanoparticles with hypericin. Each image in FIG. 4 is viewed at a different part of the respective sample. This clearly indicates the selective targeting of nanoprobe to the EGFR over expressing cancer cells.

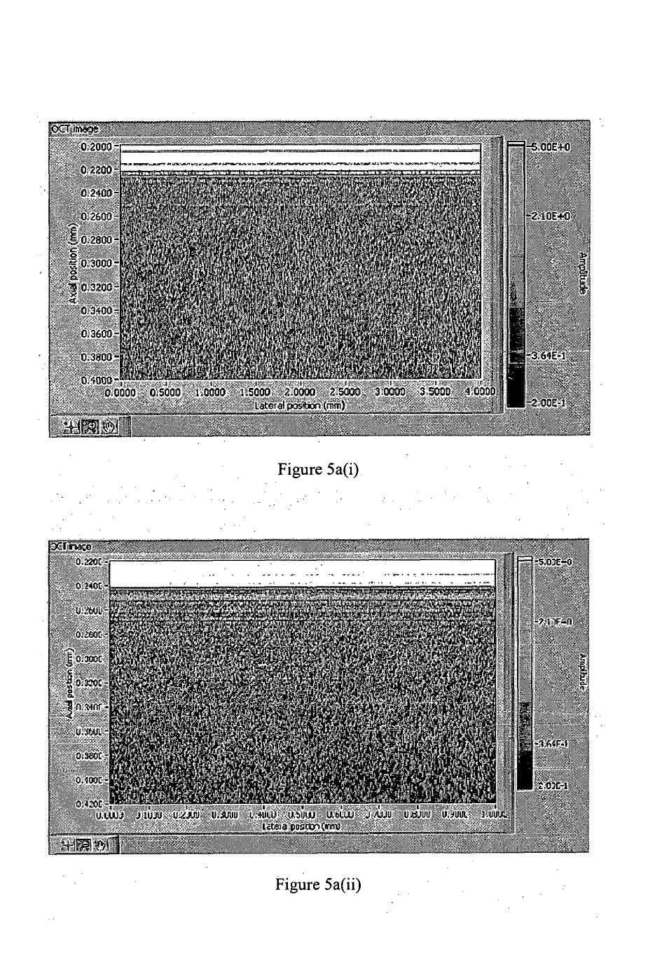

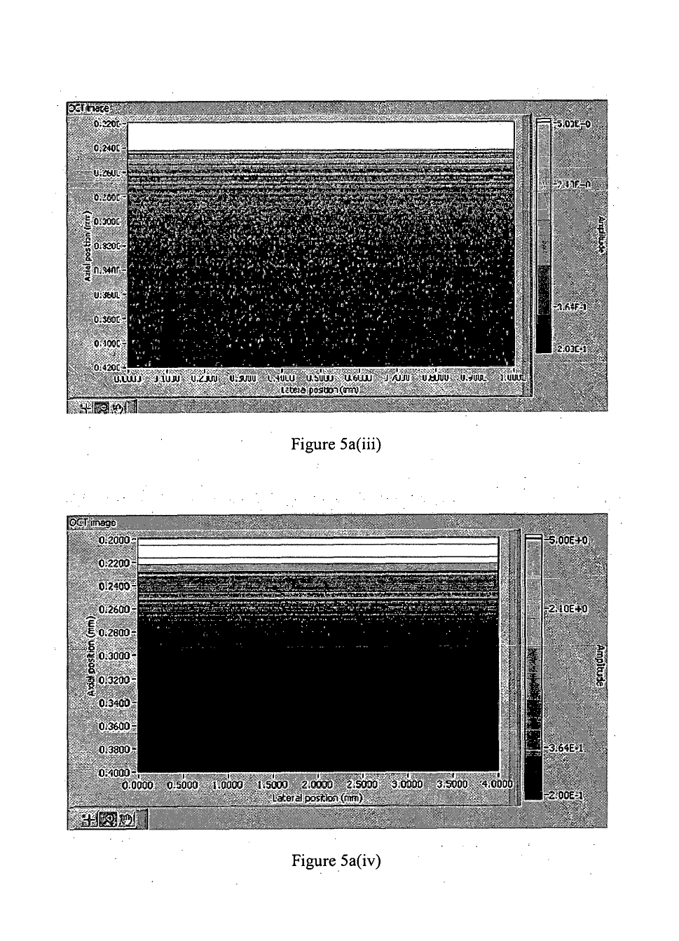

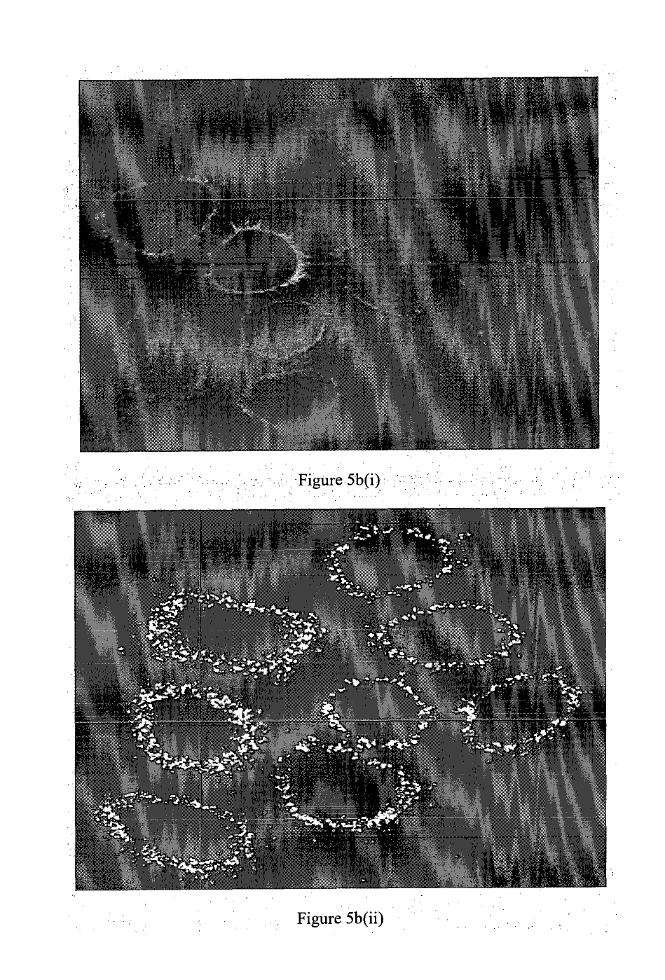

Better optical contrast in OCT imaging using nanogold constructs compared to other colloidal samples is demonstrated. FIG. 5a shows OCT m-scans of (i) colloidal suspension of nanogold; (ii) 1% Intralipid used to mimic tissue scattering; (iii) colloidal suspension of naked 302 nm silica nanoparticles (2×10¹¹ particles/ml); and (iv) saline as a negative control. The range of scanning depth shown is 200 μm with the bright signal from the top reflective layer attributed to the reflective glass slide.

The OCT m-scan of the colloidal suspension of nanogold gives a comparatively higher level of back reflectance (FIG. 5a(i)) as compared to other colloidal samples (FIGS. 5a(ii)-5a(iv)). This higher level of back reflectance would facilitate better optical contrast in OCT imaging using nanogold constructs.

FIG. 5b shows OCT contrast agent on cancer cells with nanogold hypericin with (i) EGFR fluorescence image; (ii) reflectance image of nanogold-hypericin; and (iii) combined images. Using various kinds of images, the OCT contrast agent on cancer cells with nanogold hypericin is apparent.

As another example, FIG. 5c shows OCT reflectance based confocal image of A-431 EGFR positive cancer cells incubated with anti-EGFR labelled hypericin-GNP.

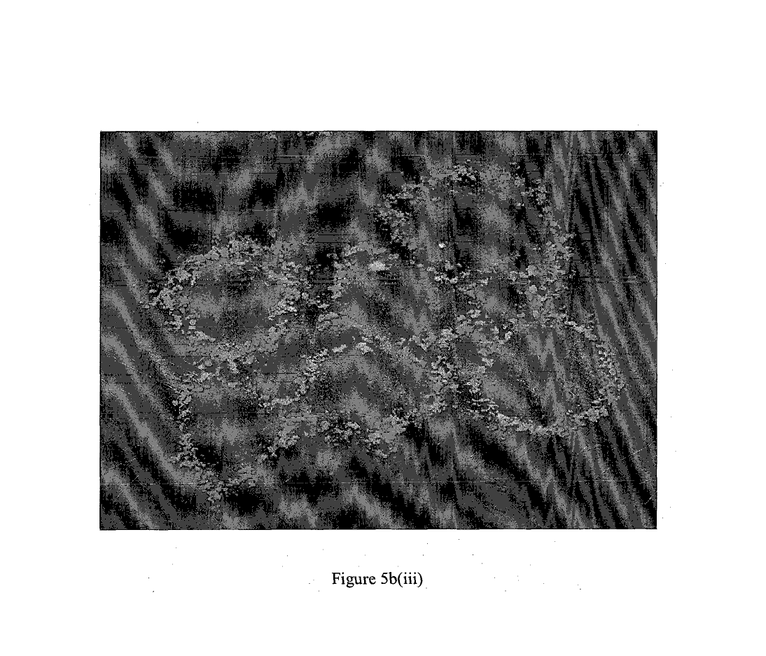

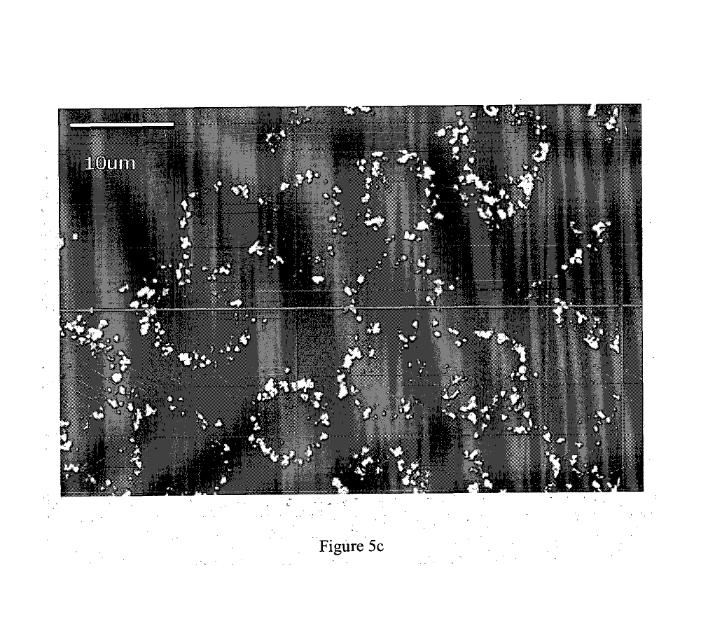

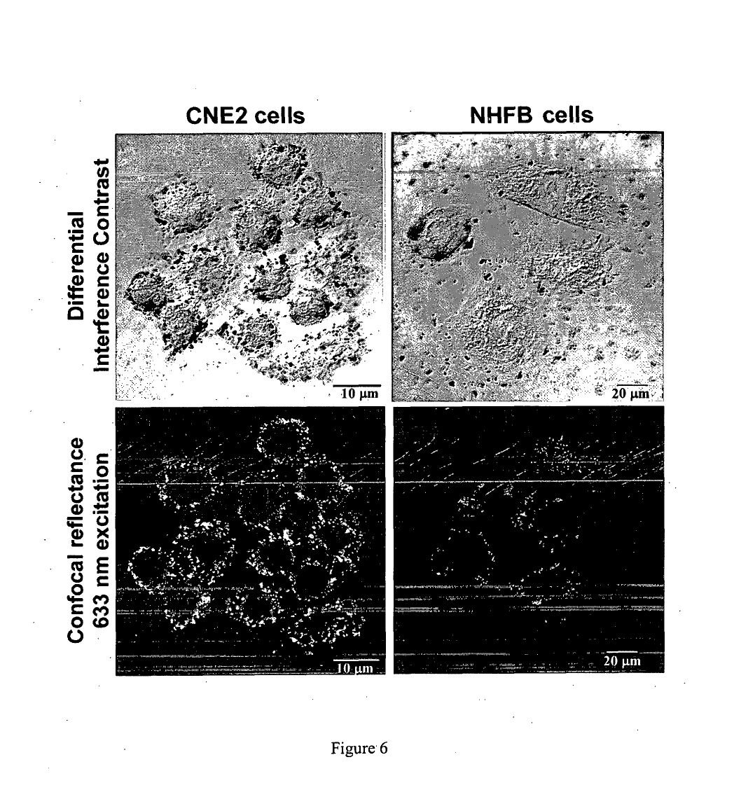

FIG. 6 shows another example of OCT reflectance based confocal images of CNE2 (Nasopharyngeal carcinoma) cancer cells compared with normal human fibroblast (NHFB) cells incubated with anti-EGFR labelled hypericin-GNP.

### Example 4

### Internalization of Hypericin-Ay-EGFR theragnostic probe by tumor cells

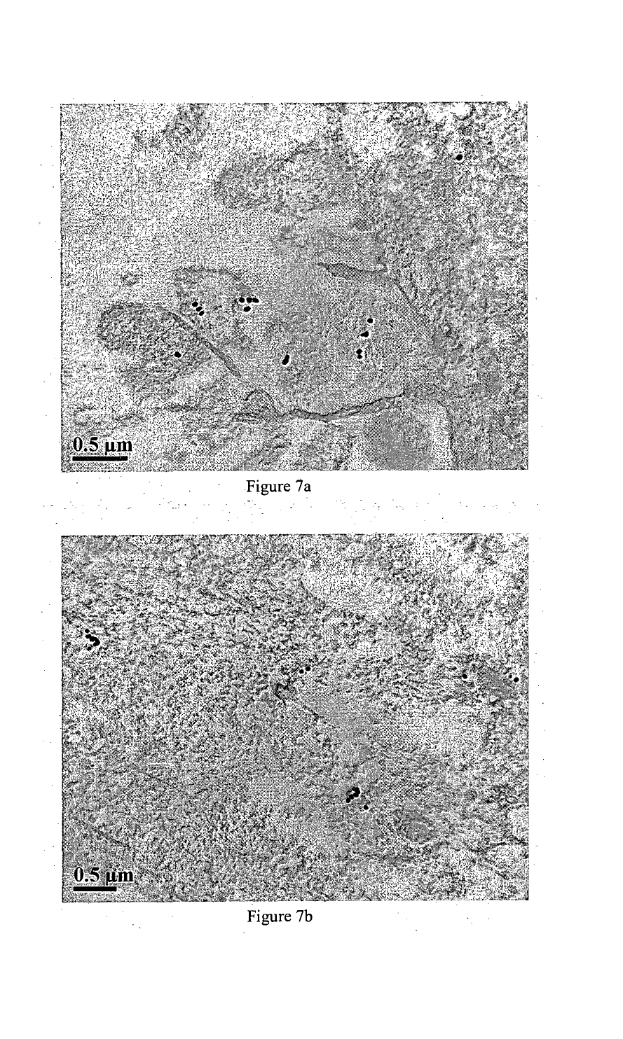

The selective internalization of the Hypericin-Au-EGFR theragnostic probe by tumor cells is studied by treatment of A-431 cells with the nanoprobe followed by TEM analysis. After about 12 hours of nanoprobe treatment, particles are found to be present in the cell cytoplasm and they appear as dark spots under TEM analysis, as shown in FIGS. 7a and 7b depicting different cell sections of A-431 cells. It is noticed that individual particles as well as clusters accumulated in the cytoplasm of the tumor cells are treated with Hypericin-Au-EGFR nanoprobe. In the case of the control (untreated cells) no such features are noticed in the TEM analysis under the same conditions.

Moreover, the Hypericin-Au-EGFR treated tumor cells shows hypercin fluorescence from the cytoplasm. This observation indicates selective internalization of Hypericin-Au-EGFR based on TEM and provides evidence for hypericin release from inside the cytoplasm. Such a probe internalization and drug release should enable the reduction of dose of the PDT drug ameliorating drug toxicity related problems in treatment.

### Example 5

### In-Vitro Uptake of Nanoprobe

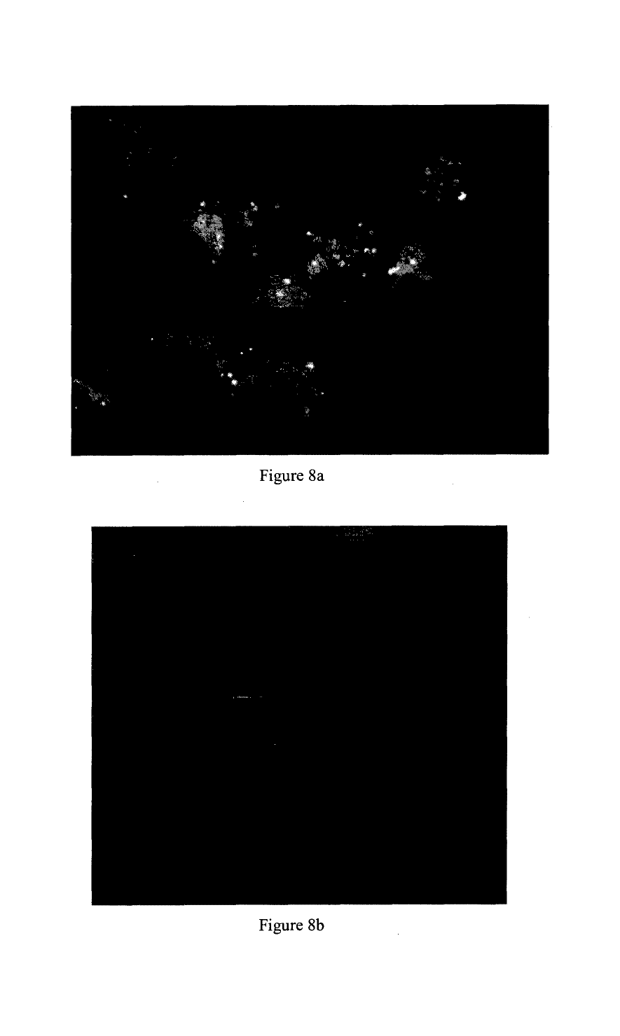

Confocal fluorescence microscopy is used to study CNE2 cells treated with 2 hypericin and A-431 cells treated with Hypericin-Au-EGFR theragnostic probe for about 6 hours under physiological conditions. This show hypericin fluorescence from within the cell in both the cases (FIGS. 8a and 8b), indicating that hypericin delivery is achieved by the Hypercin-Au-EGFR probe. This also shows that after the nanoprobe is internalized by the cell lines, hypercin is released in side the cells. This will be ideal for a probe for diagnosis and therapy.

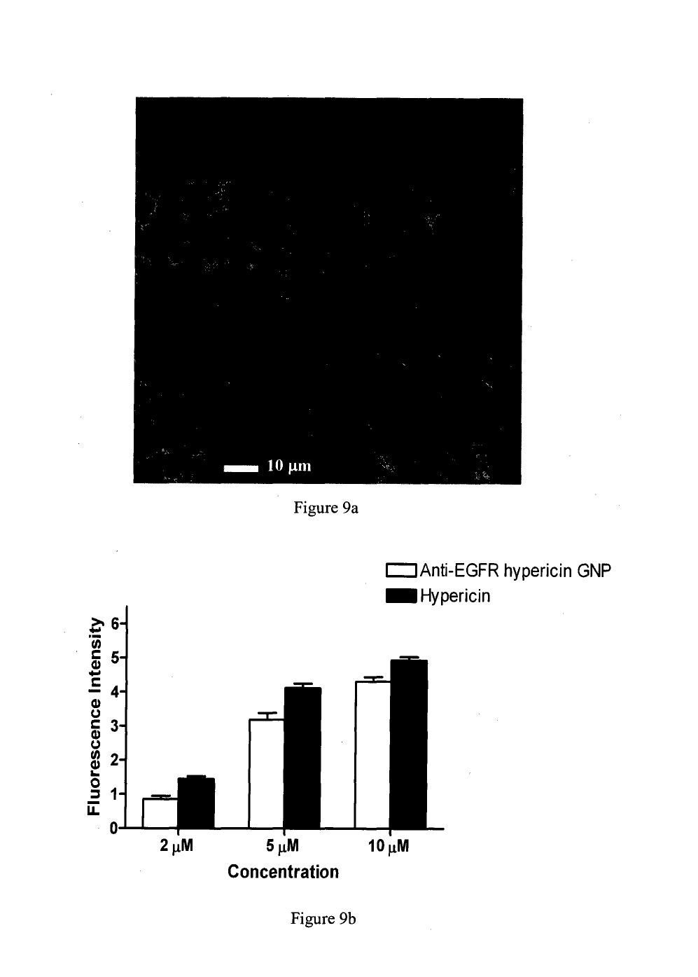

Using confocal laser scanning microscope, the in-vitro uptake of hypericin-GNP labeled with anti-EGFR after about 3 hours incubation is also assessed. In FIG. 9a, the effect of Hypericin-Au-EGFR in comparison with hypericin alone in CNE2 cell lines is studied. The cells shows strong hypericin fluorescence, which is mainly confined to the cytoplasm. The intensity of hypericin fluorescence following incubation of hypericin alone is greater compared to incubating the cells with bioconjugated hypericin GNP conjugate (FIG. 9b).

### Example 6

### Toxicity Assay

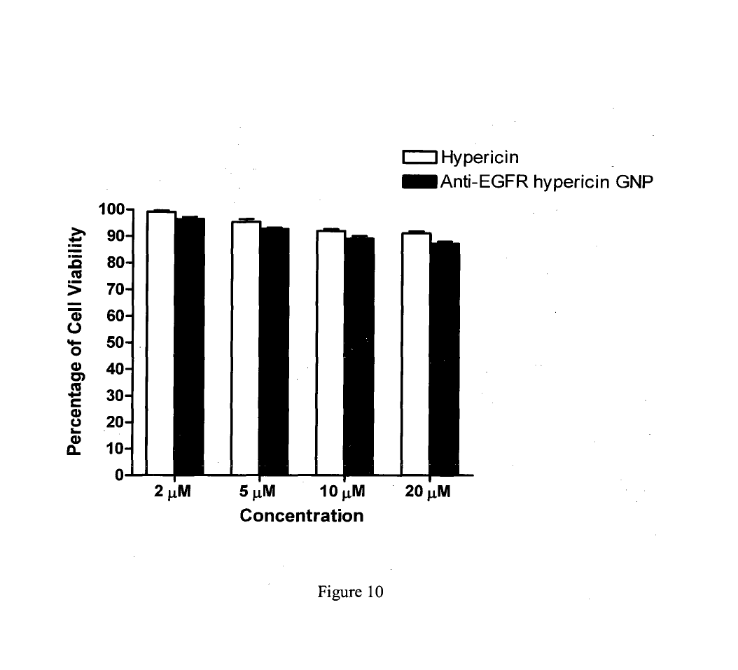

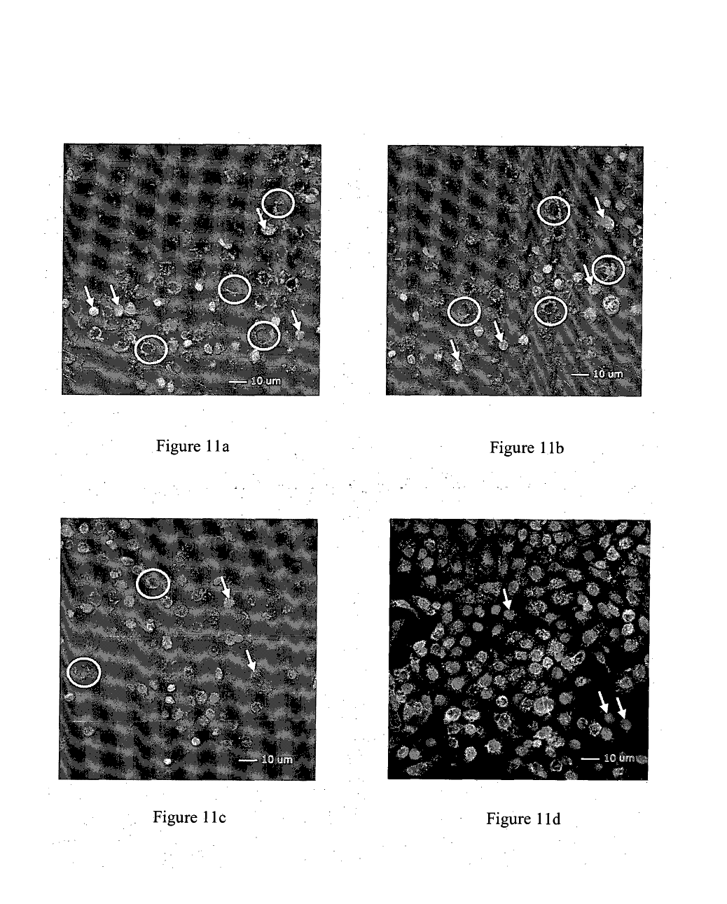

Incubation of anti-EGFR conjugated hypericin GNP with A-431 in the dark is slightly toxic when compared to incubation with hypericin alone. However, cell viability of more than 90% is observed at concentrations of up to 10 μM (FIG. 10). As the concentration of the anti-EGFR hypericin GNP increases, there is a slight decrease in the viability of the cells compared to hypericin alone.

### Example 7

### PDT Using Hypericin-Gold-EGFR Theragnostic Probe in Cellular Model

After establishing the internalization and delivery capabilities of Hypericin-Au-EGFR probe, the therapeutic potential of the probe by conducting a PDT experiment in a cellular model is examined. Hypericin-Au-EGFR probe and various amounts of hypericin are incubated with A-431 cell lines for about 12 hours and PDT is conducted using a broadband excitation using white light. Post PDT images are recorded using a confocal fluorescence microscopy with differential staining for apoptosis in green (as indicated by the white encircled area) and necrosis in red (as shown by the lighter shade indicated by the white arrow) to assess the PDT effect.

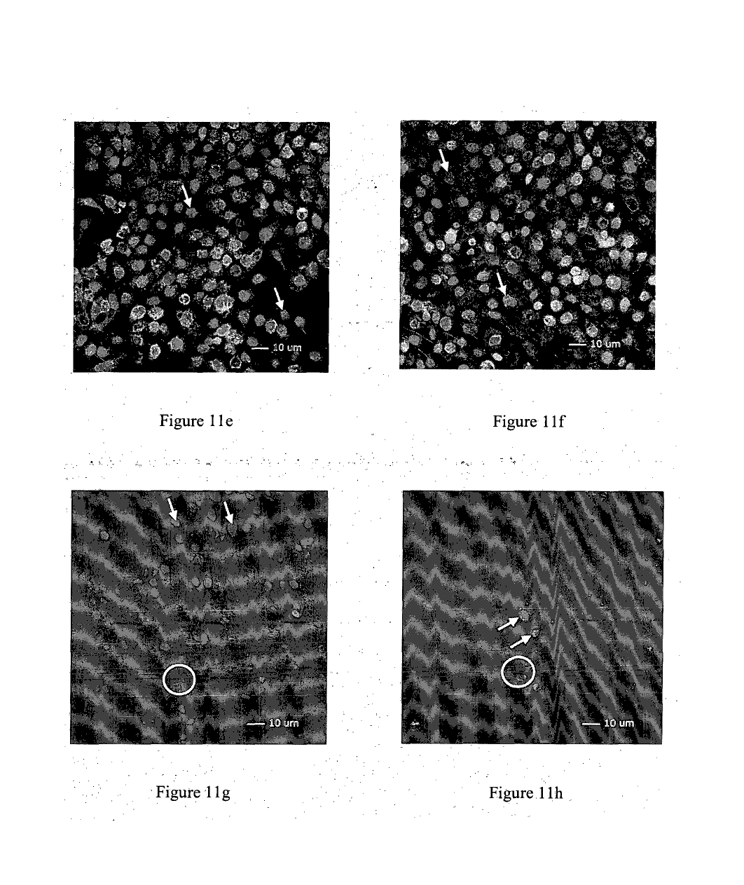

FIG. 11 shows confocal fluorescence microscopy images after exposure to light for PDT in the case of A-431 cells treated with (a) Hypericin-Au-EGFR theragnostic probe, (b) Hypericin 2 μM; (c) Hypericin 4 μM; (d) Hypericin 6 μM; (e) Hypericin 8 μM; Hypericin 10 μM; (g) A-431 cells treated with Hypericin-Au-EGFR theragnostic probe before PDT; and (h) A-431 cells control treated with no particles.

A comparison of FIG. 1la and 11b indicates Hypericin-Au-EGFR theragnostic probe induces similar amount of apoptosis as in the case 2 μM hypericin after PDT. This shows that Hypericin-Au-EGFR probe is able to deliver hypericin retaining its PDT effect. Increasing amount of hypericin caused cell death via necrosis during PDT as shown in the control experiments in FIGS. 11b to 11f. Comparison of FIGS. 11a and 11f clearly shows apoptosis induced by PDT for the Hypericin-Au-EGFR probe as opposed to necrosis as the only pathway for the cell death in the absence of PDT for the probe under the same conditions.

### Example 8

### Cell-Death Mechanism after PDT and PTT

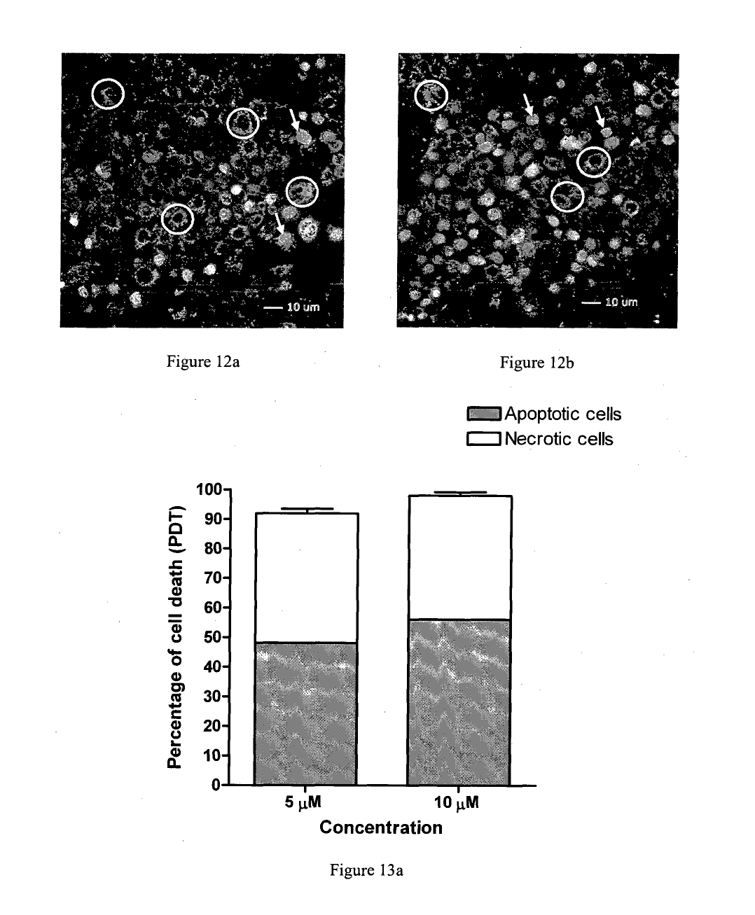

The mechanism of cell-death, both after PDT and PTT, is studied after about 3 hours incubation with EGFR-conjugated hypericin GNP and EGFR-conjugated GNP respectively. The cells undergo both apoptosis and necrosis following PDT and PTT. FIG. 12 shows confocal image of A-431 cells undergoing apoptosis (as indicated by the white encircled area) and necrosis (as shown by the lighter shade indicated by the white arrow) following (a) PDT and (b) PTT, respectively.

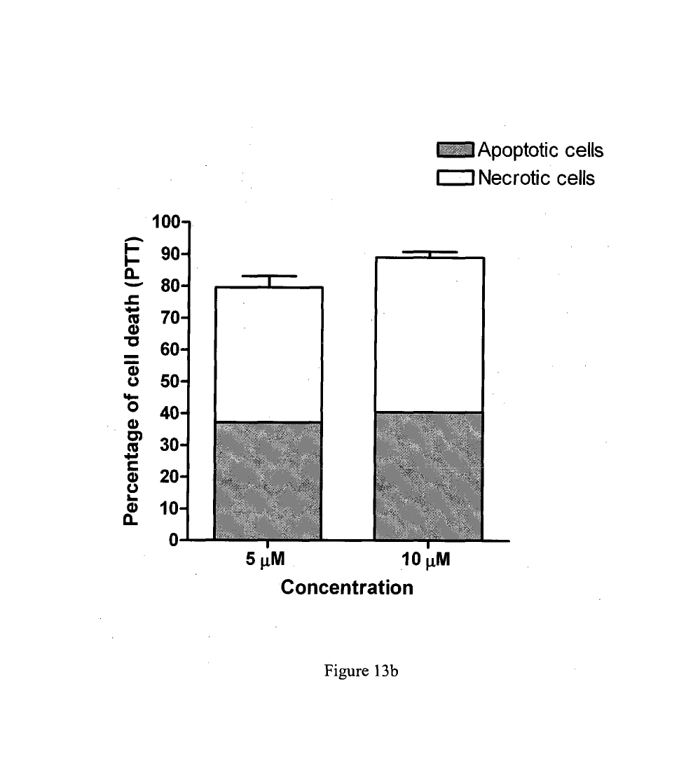

Annexin V labeled with fluorescein (FITC) stains apoptotic cells in green by binding to phosphatidylserine exposed on the outer leaflet of the cells and ethidium homodimer III stains necrotic cells with red fluorescence. The percentage of cell death is higher following PDT compared to PTT, as shown in FIGS. 13a and 13b, respectively, using different concentration of anti-EGFR conjugated hypericin GNP and anti-EGFR GNP.

### DISCUSSION

Development of a nanoprobe, combining nanotechnology and photodynamic therapy, for targeted delivery of photosensitizer and multimodality imaging and therapy is carried out. Nanoparticles represent emerging photosensitizer carriers that may overcome most of the shortcomings of conventional photosensitizers, for example, for use in PDT.

Gold nanoparticles (GNPs) are spherical metallic nanoparticles for biological use due to their the small size, creating a large surface area to volume ratio. Secondly, their size may be optimised for efficient intravascular transport and accumulation inside tumor beds for selective tumor targeting and drug delivery. Thirdly, strong absorption and scattering of GNP provides an opportunity for their use in contrast enhanced optical imaging.

In various embodiments, GNPs are used as delivery vehicles for targeted delivery of hydrophobic photosensitizer, hypericin. The hydrophobic property of hypericin is used to attach the GNP by non-covalent interactions and in accordance to various embodiments, by hydrophobic interactions. This at least minimizes or eliminates problems of altered photosensitizer activity due to chemical modification caused by covalent attachment. Furthermore, with non-covalent interactions, the release of the photosensitizer from the GNP may be easier when compared covalent interactions.

Gold nanoparticles with their unique surface plasmon properties may serve as contrast agents as well as delivery vehicles for the delivery of photosensitizer. Moreover, capping the gold surface with polyethylene glycol, the surface properties may be tuned to match the requirement. In various embodiments, PEG reagents with two different functionalities are used. Carboxy PEG are used to attach antibody and methoxy PEG is used for complete gold surface coverage to prevent non specific interactions with the cellular components as well as for longer circulation times. Carboxyl group of the carboxy PEG upon activation with EDC and NHS forms O-acylisourea as the active ester that reacts with amino groups on the antibody to give the covalently tagged antibody on gold surface.

To achieve targeted delivery of hypericin loaded GNPs to tumor cells, the GNPs are conjugated with anti-EGFR antibody. The epidermal growth factor receptor (EGFR) signaling pathway plays an important role in the regulation of cell proliferation, survival, and differentiation. Amplification and overexpression of EGFR is found in many cancer types, which provides an opportunity for designing receptor-targeted approaches for cancer detection and treatment. EGFR proves be a good mediator for the targeted drug delivery due to its differential over-expression in tumor cells coupled with the fact that EGFR-antibody complex may be internalized by the cells. Targeting of antibody conjugated GNPs is for bioimaging applications and this technique is adopted for the targeted delivery of the photo sensitizer to the tumor cells reducing its toxicity.

To determine the sub-cellular localization of the bioconjugated GNPs in EGFR overexpressing A-431 cells, the particles are incubated for about 3 hours at about 37° C. and visualized using a dark field reflectance microscope. It is found that at about 3 hours, the bioconjugated GNP are incorporated in the cells and they scatter strong yellowish light. Examination reveals that they are primarily attached to the cell surface via the receptor specific targeting. It is shown that the EGFR-mediated internalization of the targeted nanoparticles in tumor cells increases the retention time and amount of the nanoparticles inside the tumor mass. Moreover, non-targeted nanoparticles easily accumulate in the liver and spleen after systemic delivery accounting for their toxicity. Thus receptor mediated drug targeting may reduce systemic toxicity due to the requirement of lower concentration of the nanoprobe for effective therapy. The in vitro dark toxicity results also show that there is no significant difference between hypericin and anti-EGFR conjugated hypericin. GNPs at concentrations ranging from 2-20 μM. Although there is a slight decrease in the viability of the cells with the increase in concentration of anti-EGFR hypericin GNP, this is not significant when compared with the toxicity of hypericin at the given concentration.

Confocal laser scanning microscope is used to assess the in vitro uptake of hypericin in the bioconjugated nanocontruct in A-431 cells. Hypericin fluorescence intensity is analysed following incubation with different concentration of anti-EGFR hypericin GNP ranging from 2-10 μM. It is observed that the hypericin is concentrated predominantly in the cytoplasm, with negligible fluorescence in the nuclear region. However, A-431 cells incubated with hypericin alone exhibit about 1.1 to 1.7 folds higher fluorescence intensity than those incubated with anti-EGFR hypericin GNP. This may be attributed to the relatively large size of the EGFR antibody, which limits the number of ligands that may be linked to the surface of a nanoparticle and impedes intracellular distribution of the complex leading to the reduced availability of hypericin loaded GNPs to the cells. The loading of hypericin in the nanoprobe may be optimized.

As an alternative, a single-chain anti-EGFR antibody (ScFv EGFR) which is smaller than 20% of an intact antibody may be used to replace bulky anti-EGFR antibodies. The resulting antibody fragment (25 to 28 kDa) may provide a much smaller targeting ligand, maintaining a high binding affinity and specificity.

To evaluate the phototoxicity and mode of cell death after PDT and PTT, A-431 cells are incubated using anti-EGFR hypericin GNP and anti-EGFR-GNP respectively and irradiated at a fluence of 1 J/cm². Cell viability dramatically decreases both after PDT and PTT. However, cell death following PDT is about 1.2 folds higher than that following PTT. This may be due to the synergistic effect of the phototoxicity due to the photosensitizer hypericin and hyperthermia caused by the GNPs. When GNPs absorb light the free electrons in the gold particles are excited. Upon interaction between the electrons and the crystal lattice of the gold particles, the electrons relax and the thermal energy is transferred to the lattice. Subsequently, the heat from the gold particles is dissipated into the surrounding environment. Cells are very sensitive to small increases in temperature. When bioconjugated GNPS are heated by absorption of light, the temperature of cells in the vicinity of the particles is raised thus inducing selective killing of the cells. Thus, though PTT alone may cause severe damage to cells, the combination treatment is found to be much more effective. However the mode of cell death does not significantly differ greatly among the two treatment modes, as there is a mixture of apoptotic cells as well as necrotic cells following both treatments. The PDT and PTT conditions such as the irradiation time, light dosage and concentration of GNPs may be optimized to achieve maximum cell death.

In addition to their application in PDT and PTT, anti-EGFR conjugated hypericin GNPs may be used as multimodality imaging of cancer cells. Successful adsorption of photosensitizer by sonicating photosensitizer and gold nanoparticles is evidenced by SERS spectra. Also, intrinsic Raman activity of the natural photosensitizer hypericin SERS is used as one of imaging tool in a multimodality platform. Such highly multifunctional nanoprobes with imaging and therapeutic capabilities allows for early detection and therapy of cancer.

This is possible by the inherent properties of GNPs by virtue of their optical properties and surface plasmon resonance (SPR). When stimulated by light, SPR of gold strongly enhances the Raman scattering of adjacent molecules by about 6 orders of magnitude creating SERS effects. Hypericin molecule by itself may act as a SERS reporter, due to the high SERS cross-section of hypericin molecule owing to its planar geometry and hydrophobicity. Hypericin retaining SERS activity even after pegylation and bioconjugation of the GNPs are observed. This property of the bioconjugated hypericin nanogold construct may be utilized to develop non-invasive biosensing probes specific to cancers overexpressing EGFR.

Besides this feature, GNPs also have high scattering cross-section in the red (or lighter shaded) region of the spectrum. This property is crucial for development of contrast agents for optical imaging in living organisms because light penetration depth in tissues dramatically increases with increasing wavelength. Agglutinated particles have much higher scattering cross-section than individual particles. This property may be used for vital optical imaging such as Optical Coherence Tomography (OCT), which is a non-invasive optical imaging technique that produces cross-sectional images of tissue with a high spatial resolution of about 10 to 20 μm. Thus, based on these contrast enhancement properties of GNP, anti-EGFR conjugated hypericin GNP may be used for highly sensitive and specific molecular imaging of cancers overexpressing EGFR via the OCT imaging technique.

Apart from the optical properties of GNP, the photosensitizer hypericin itself may be employed as fluorescent probes for bioimaging applications such as three dimensional (3D) laser confocal fluorescence endomicroscopy. This is a relatively new optical technique that offers in vivo confocal imaging of tissue structures from surface to subsurface layers down to a few hundred micrometers, utilizing fluorescent probes such as fluorescein, hypericin and 5-ALA (aminolevulinic acid). Thus bioconjugated hypericin loaded GNPs may be developed as multimodality theragnostic probes.

Design of adequate photosensitizer delivery systems is critical for improving the outcome and acceptability of PDT in a clinical context. The use of GNPs as carriers of photosensitizers is an approach which not only offers improved trafficking of hydrophobic photosensitizers into the cells, but also improves the efficacy of PDT due to the photothermal effect. In essence, a combined PDT and PTT treatment modality may be a more effective treatment strategy compared to conventional PDT. Furthermore, targeted delivery of the photosensitizers using antibodies specific to certain tumor cell types, provides an added advantage of high selectivity with low toxicity, rendering minimal damage to the normal tissues. In addition to this, such nanoprobes may also be used as multifunctional molecular optical diagnostic probes for cancer imaging and phototherapeutics. That is, combining advances in photodynamic therapy and nanotechnology may lead towards improved multimodality therapeutics and imaging. Techniques for imaging of cancer in optical multiple modalities, using a single agent in a single session, are developed, and this technique is known as ‘optical multimodality imaging’. For target-specific imaging probe, various targeting ligands, such as photosensitizers, small molecules, antibodies, peptides and aptamers may be used in developing cancer imaging probes that are highly target specific and biocompatible.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A nanoparticle loaded with a light sensitive molecule, wherein the nanoparticle is a colloidal gold nanoparticle and
the light sensitive molecule is non-covalently adsorbed to the surface of the nanoparticle.

2. The nanoparticle as claimed in claim 1, wherein the non-covalent interaction is hydrophobic interaction.

3. The nanoparticle as claimed in claim 1 or 2, wherein the light sensitive molecule is a photosensitizer.

4. The nanoparticle as claimed in claim 3, wherein the photosensitizer is selected from the group consisting of hypericin, Photofrin, Visudyne, aminolevulinic acid-induced protoporphyrin IX (ALA-induced Pp IX), Foscan, Chorin e6, mono-L-aspartyl chlorin e6 (NPe6) or Laserphyrin, propiophenone, anthrone, benzaldehyde, butylophenone, 2-naphthylphenylketone, 2-naphthaldehyde, 2-acetonaphthone, 1-naphtylphenylketone, 1-acetonaphthone, 1-naphtho aldehyde, fluorenone, 1-phenyl-1,2-propane dione, benzoethrile, acetone, biacetyl, acridine orange, acridine, Rhodamine-B, eosine, fluorescein, Silicon Phthalocyanine Pc 4, m-tetrahydroxyphenylchlorin (mTHPC), Allumera, Levulan, Metvix, Amphinex, Azadipyrromethenes, and a mixture thereof.

5. The nanoparticle as claimed in claim 3 or 4, wherein the photosensitizer produces singlet oxygen upon activation with a light source.

6. The nanoparticle as claimed in any one of claims 1 to 5, wherein the gold nanoparticle is about 10 nm to about 1000 nm in size.

7. The nanoparticle as claimed in any one of claims 1 to 6, wherein the gold nanoparticle is about 40 nm in size.

8. A nanoprobe comprising the nanoparticle as claimed in any one of claims 1 to 7 and further comprising a targeting moiety covalently coupled to the surface of the nanoparticle.

9. The nanoprobe as claimed in claim 8, wherein the targeting moiety is selected from the group consisting of a small molecule, an antibody, an antigen, an affibody, a peptide, an aptamer, a cell surface receptor ligand, a nucleic acid, a fibronectin, a protein, a fusion protein, a peptide, a biotin, and a conjugate thereof, and a chemical moiety.

10. The nanoprobe as claimed in claim 8 or 9, wherein the targeting moiety is covalently coupled to the surface of the nanoparticle by a linking moiety.

11. The nanoprobe as claimed in claim 10, wherein the linking moiety is polyethylene glycol (PEG) or a derivative thereof.

12. The nanoprobe as claimed in claim 11, wherein the PEG has a molecular weight ranging from about 1000 to about 8000.

13. The nanoprobe as claimed in any one of claims 8 to 12, wherein the nanoprobe is a multimodal optical nanoprobe.

14. A pharmaceutical formulation comprising a nanoparticle as claimed in any one of claims 1 to 7 or a nanoprobe as claimed in any one of claims 8 to 13.

15. A method of preparing a nanoparticle as claimed in any one of claims 1 to 7, comprising the steps of:
providing a colloidal gold nanoparticle; and
non-covalently adsorbing a light sensitive molecule to the surface of the gold nanoparticle such that the light sensitive molecule is immobilized on said surface.

16. The method as claimed in claim 15, wherein the step of non-covalent adsorbing comprises:
adding a solution of the light sensitive molecule into a solution comprising the colloidal gold nanoparticle; and
sonicating for about 2 hours at a temperature of about 20° C.

17. The method as claimed in claim 15 or 16, further comprising the step of functionalizing the nanoparticle with a targeting moiety.

18. The method as claimed in claim 17, wherein the targeting ligand is selected from the group consisting of a small molecule, an antibody, an antigen, an affibody, a peptide, an aptamer, a cell surface receptor ligand, a nucleic acid, a fibronectin, a protein, a fusion protein, a peptide, a biotin, and a conjugate thereof, and a chemical moiety.

19. The method as claimed in claim 17 or 18, wherein the functionalizing step comprises modifying the surface of the gold nanoparticle with a linker moiety and coupling the targeting moiety to the linker moiety.

20. The method as claimed in claim 19, wherein the linker is polyethylene glycol (PEG) or a derivative thereof.

21. The method as claimed in claim 20, wherein the PEG is carboxy PEG.

22. The method as claimed in claim 21, wherein the functionalizing step further comprises:
activating the carboxyl group of the carboxy PEG with N-(3-dimethylaminopropyl)-N′ ethylcarbodiimide (EDC) and N-Hydroxysuccinimide (NHS) to form O-acylisourea as an active ester; and
reacting the active ester with amino groups on an antibody to covalently couple the antibody to the surface of the gold nanoparticle to form a bioconjugated gold nanoparticle.

23. The method as claimed in claim 22, wherein prior to the reacting step, the method further comprises adding a stabilizer.

24. The method as claimed in claim 23, wherein the stabilizer comprises sodium salt.

25. The method as claimed in claim 24, wherein the sodium salt is sodium azide.

26. The method as claimed in claim 22 or 23, wherein after the activating step and prior to the reacting step, the method further comprises performing dialysis to remove unreacted EDC and NHS, and/or to remove the stabilizer.

27. The method as claimed in claim any one of claims 22 to 26, wherein the antibody is an anti-EGFR (Epidermal Growth Factor Receptor) antibody or an anti-Her 2Neu (Human Epidermal growth factor Receptor 2) antibody.

28. An imaging method, comprising:
administering a nanoprobe as claimed in any one of claims 8 to 13 to a subject; and
collecting imaging data of the subject or part of the subject with optical multimodality imaging.

29. The imaging method as claimed in claim 28, wherein the optical multimodality imaging is in vivo imaging or ex vivo imaging.

30. The imaging method as claimed in claim 28 or 29, wherein the optical multimodality imaging is selected from the group consisting of magnetic resonance imaging, ultrasound imaging, confocal fluorescence endomicroscopy, optical coherence tomography (OCT), Surface Enhanced Raman Spectroscopy (SERS) and a combination thereof.

31. The imaging method as claimed in any one of claims 28 to 30, wherein the portion of the subject comprises a tumor cell.

32. The imaging method as claimed in claim 31, wherein the tumor cell is a cancer cell or a cancerous cell line.

33. A method for determining a photodynamic therapy regimen for a subject comprising determining the therapy regimen based on imaging data collected with optical multimodality imaging after a nanoprobe as claimed in any one of claims 8 to 13, or a nanoparticle prepared by the method as claimed in any one of claims 15 to 27 has been administered to the subject.

34. The method as claimed in claim 33, wherein the photodynamic therapy program is coupled with photothermal effects rendered by plasmonic heating effects of the nanoparticle.

35. Use of intrinsic Raman activity of a light sensitive molecule for Surface Enhanced Raman Spectroscopy (SERS) based imaging, wherein the light sensitive molecule is a photosensitizer comprised in a nanoparticle as claimed in any one of claims 1 to 7, or a nanoprobe as claimed in any one of claims 8 to 13, or a nanoparticle prepared by the method as claimed in any one of claims 15 to 27.

36. A method of treating cancer in a subject comprising administering a nanoprobe as claimed in any one of claims 8 to 13; and performing photodynamic therapy on the subject.

37. The method as claimed in claim 36, wherein the photodynamic therapy comprises incubating the nanoprobe with a tumor cell to allow internalization in the cell; and upon internalization, illuminating the cell to cause cell death by reactive oxygen species generated by the light sensitive molecule of the nanoprobe.

38. The method as claimed in claim 37, wherein the light sensitive molecule is a photosensitizer.

39. The method as claimed in any one of claims 36 to 38, wherein the method is based on targeting of a receptor in the cell selected from the group consisting of an integrin, a somatostatin receptor, an epidermal growth factor receptor (EGFR), a Her-2/neu receptor, a glucose transporter (GLUT), a folate receptor, and a steroid receptor.
